(19) United States
(12) Patent Application Publication
(10) Pub. No.: US 2014/0024977 A1
(43) Pub. Date: Jan. 23, 2014

(54) SOFT TISSUE THERAPY DEVICE AND METHOD OF USE THEREFOR

(71) Applicant: TherapyCare Resources, Inc., Indianapolis, IN (US)

(72) Inventors: MICHAEL I. ARNOLT, Indianapolis, IN (US); James W. Daniel, Indianapolis, IN (US)

(73) Assignee: TherapyCare Resources, Inc., Indianapolis, IN (US)

(21) Appl. No.: 13/629,583

(22) Filed: Sep. 27, 2012

Related U.S. Application Data
(60) Provisional application No. 61/540,262, filed on Sep. 28, 2011.

(51) Int. Cl. A61N 1/26

(52) U.S. Cl. CPC A61N 1/26
USPC 601/21

(57) ABSTRACT

A therapy tool system includes a rigid therapy tool for passing across a patient's skin for loosening subcutaneous tissue. The rigid tool includes an electrically conductive portion engageable with a patient's skin. A current source is provided for providing an electric current. A connector member is provided that is selectively attachable and removable from the rigidtool. The tool member is coupled to the current source for conducting current between the current source and the tool. The rigid tool includes a conductive path for conducting current from the connector to the electrically conductive portion of the tool, to enable current delivered to the connector portion from the current source to be conducted to the electrically conductive portion of the tool and transferred from the tool through the patient's skin to subcutaneous patient tissues.

### CLAIM OF PRIORITY

The instant application claims benefit of priority to Arnolt and Daniel, U.S. Provisional Patent Application No. 61/540,262 that was filed on 28 Sep. 2012, and which is fully incorporated herein by reference.

### I. TECHNICAL FIELD OF THE INVENTION

The present invention relates to medical devices and processes, and more particularly to methods for performing therapy on a subject, and tools for performing therapy on a patient.

### II. BACKGROUND OF THE INVENTION

Inflammation of soft tissue areas (or soft tissue dysfunction) of the human body may occur in many ways. For example, inflammation may occur as the result of a major trauma, such as surgery, or as the result of repeated micro-trauma, such as a repetitive strain injury, overtraining or an accident. The body responds to such inflammation by forming fibrotic adhesions or scar tissue, as an unavoidable by-product of the healing process. The scar tissue forms in soft tissue areas of the body, such as muscles, tendons and ligaments, and in the area between the muscle and the connective tissue (fascia).

As scar tissue builds up, it prevents the muscles, tendons and ligaments from properly lengthening and contracting, thereby resulting in lost/reduced range of motion, pain and decreased stability. Additionally, the build-up of scar tissue generally causes pain in the affected area/joint and surrounding areas. For a person to return to full function, or better function, it is desirable to loosen or remodel the scar tissue (tissue dysfunction), so the joint/soft tissue may achieve a greater level of mobility and performance.

Scar tissue is removed or remodeled by a process known as soft tissue therapy, which involves use of the clinician's hand to manually massage the skin over the affected soft tissue areas to release scar tissue adhesions and regain lost resting length in the tissue. This type of massage includes cross-frictional massage, deep muscle massage and rolfing.

One problem associated with manual massage of soft tissue areas is the difficulty in applying the appropriate amount of manual pressure. In some instances, too much pressure may be exerted by the clinician (or other medical practitioner) on some soft tissue areas, thereby causing unnecessary discomfort to the patient. In other instances in which hardened scar tissue has built up on tendons and ligaments near bone surfaces, the clinician may not be able to apply sufficient pressure with his or her hands to provide an effective treatment. Also, it is frequently difficult for the clinician to manually locate or detect scar tissue with sufficient specificity using his or her hand. Furthermore, it has been found that performing manual massage for an extended period of time may result in injuries, such as tendinosis, to the hand and wrist of the clinician.

To overcome the aforementioned problems with manual therapy, a soft tissue therapy process was invented by David A. Graston, that employed a set of tools in order to perform the therapy process. This therapy method and the accompanying tools are disclosed in Graston, U.S. Pat. Nos. 5,231,977; 5,366,437; 5,441,478; and 5,707,346, the disclosures of which are all incorporated herein by reference. The Assignee of the present invention has promoted and licensed the therapy methods and the tool discussed therein, and has met with great commercial success.

Nonetheless, room for improvement exists in the use of the tools and methods described in the above-referenced patents. In particular, room for improvement exists to increase the efficacy of the soft tissue therapy by the use of electrical stimulation, along with the manual therapy stimulation performed by the tools and the clinician.

Electrical stimulation therapy utilizes electrical current which is passed through a biological system to produce physiochemical and physiological effects on that system. Electrical stimulation therapy (“electro therapy”) is used in the treatment of a variety of debilitating conditions, and is frequently used in soft tissue therapy. For example, electro therapy has been used extensively in pain management programs, muscle strengthening, iontophoresis, edema reduction, and in the stimulation of denervated muscle, among other uses.

Each waveform generated by the source of the electrical current has certain aspects that are well suited for obtaining a particular desired physiological response. By varying the particular waveform, the therapist attempts to optimize the results of the treatment by matching the particular condition to be treated with the waveform that most effectively treats the particular condition. For example, classic, or Quadpolar Interferential, is believed to be optimal for sensory stimulation. Symmetric, square-wave biphasic current is believed optimal for motor-fiber stimulation. Monophasic current may be used for wound care.

Additionally, a particular pulse rate within each wave form may be selected for further optimization. In general, low pulse rates (0-10 Hz) are believed to be superior for more chronic problems, whereas higher pulse rates (80-200 Hz) are believed superior for the treatment of acute problems. For example, direct current is believed to be the most effective waveform for treatment with iontophoresis, and also for the stimulation of denervated muscle. In contrast, high voltage pulsed galvanic (HVPG) waves are preferred for use in edema reduction, pain management and muscle reeducation.

Physical therapists and other clinicians have found electro therapy to be an effective tool in the treatment of inflammation of soft tissue areas of the body. In particular, physical therapists have found electro therapy to be an effective tool, when performed in conjunction with the tools disclosed above in the various Graston patents.

In this regard, the reader's attention is directed to Graston, U.S. Pat. No. 5,707,346 and Graston, U.S. Pat. No. 6,126,620. The Graston '346 and '620 patents both relate to a method and system for performing soft tissue massage that involve a tool including a handle portion and a skin engaging portion. This skin engaging portion non-invasively engages the skin to allow the user to locate fibrous adhesions that are attached to the underlying soft tissue areas.

Although the rules and methods described in the aforementioned Graston patents have provided a highly efficacious and commercially well-accepted advance in the therapeutic arts, when compared to the prior art, room for improvement still exists. In particular, room for improvement exists in providing a massage tool for providing soft tissue massage that can be coupled to an electrical source to provide electro therapy to the patient while using the massage tool that includes an improved connector mechanism for coupling the electrical source to the tool.

Therefore, one object of the present invention is to provide such a connection system for connecting a source of electricity to massage tools.

### III. SUMMARY OF THE INVENTION

In accordance with the present invention, a therapy tool system is disclosed. The therapy tool system comprises a rigid therapy tool for passing across a patient's skin for loosening subcutaneous/dysfunctional tissue. The tool includes an electrically conductive portion engageable with a patient's skin. A current source is provided for providing an electric current. A connector member is provided that is selectively attachable and removable from the tool. The tool member is coupled to the current source for conducting current between the current source and the tool. The rigid tool includes a conductive path for conducting current from the connector to the electrically conductive portion of the tool; thus, enabling current to be delivered to the connector portion from the current source to be conducted to the electrically conductive portion of the tool and transferred from the tool through the patient's skin to subcutaneous patient tissues.

Preferably, the therapy tool connector member includes a sleeve portion for receiving the rigid tool, and a clamp for a fixed, but removable coupling from the connector to the tool member, wherein the subcutaneous tissue comprises scar tissue underlying patient soft tissue, all of which is disposed subcutaneously.

In a preferred embodiment, the sleeve portion can include a relatively wide portion for receiving the rigid tool, and a relatively narrower portion, so as to be adaptable to various sizes. The relatively narrower portion is partially defined by shoulders for limiting the movement of the tool received in the relatively wider portion.

In another preferred embodiment, the connector member comprises a magnetic member that is capable of being magnetically coupled and electrically coupled to the rigid tool. The magnetic connector member can include a plug receptacle for receiving a plug. The current source can include a plug member capable of being coupled to the plug receptacle.

Additionally, the rigid tool can comprise a first rigid tool and a second rigid tool, wherein the connector is movable between the first rigid tool and the second rigid tool. The connector includes a body that defines a sleeve portion and a clamp movable between a tool engaging position and a tool releasing position, wherein the sleeve is sized and configured to receive each of the first and second rigid tools, and the clamp is sized and positioned to engage each of the first and second rigid tools, so that the connector member and current source can be transferred between the first and second rigid tool.

One feature of the present invention is that the connector is selectively removably attachable to the rigid tool member. This feature has the advantage of enabling a particular tool, or tools, to be used both in an electro stimulation type therapy, and also in a conventional soft tissue therapy, without electrical stimulation. Although a prior art tool is capable of being used in a non-electrical stimulation therapy mode, through the user not supplying electricity to the tool, known prior art electro stimulation tools have the disadvantage of having a wire connected to them in a relatively permanent manner, e.g. via a weld that provided no good vehicle for enabling the user to move the electrical conductor between tools. Additionally, this permanently connected wire made the tool more difficult to use, and less easy to grip and manipulate for all soft tissue treatment for which it is intended, than a tool that did not have the wire and connector permanently connected to it.

Another advantage provided by the selected connection of the connector of the present invention is that a single electrical stimulation source can be used with a wide variety of tools. This provides convenience to the user, along with a significantly less cluttered work space. For example, a user at a particular patient station can employ a single electrical control device that is coupled to a single electrical wire and plug. That single connector-wire plug combination can then be applied to a variety of tools, depending upon the user's need and choice of tools for a particular therapy. By making the connector adaptable for use with a wide variety of tools, the user need not have to deal with added confusion and clutter caused by a plurality of wires and permanent welded attachment that would have otherwise be required with a known prior art system.

Another feature of the present invention is that the coupler provides for a quick coupling and de-coupling of the connector to the tool. This quick coupling helps to make the user more efficient, by not causing undue delay by switching the connector between tools, or removing or adding a connector to a tool.

An additional feature of the present invention is that it can help to reduce the cost to a clinic, and cost of the tools, since the ability of the connector to quickly connect and de-connect to a plurality of tools, reduces the need for the user to have a first set of non-electrically stimulated tools, and a second set of electrical stimulation therapy tools.

These and other features and advantages of the present invention will become apparent to those skilled in the art upon a review of the drawings and detailed description of the present invention presented below, that is believed to disclose the best mode of practicing the invention known currently to the Applicants.

### IV. IN THE DRAWINGS

FIG. 1 is a perspective, partly schematic view of prior art tool of the present invention being used to apply tissue therapy on a knee of a patient;

FIG. 2 is a top view of a prior art electro therapy soft tissue massage tool;

FIG. 2A is a side, exploded view of another prior art electro therapy soft tissue massage tool

FIG. 3 is a side view of a soft tissue massage therapy tool to which is coupled a coupler and electrical power source of the present invention;

FIG. 4 is a top view of the soft tissue massage therapy tool of FIG. 3;

FIG. 5 is an enlarged sectional view taken along lines 5-5 of FIG. 3;

FIG. 6 is an enlarged sectional view taken along lines 6-6 of FIG. 4;

FIG. 7 is a perspective view of a tool clamping type electrical connector of the present invention;

FIG. 8 is a side view of the connector member of the present invention showing the clamping screw member in the raised or disengaged position;

FIG. 9 is a side view of the connector member of the present invention of FIG. 8, showing the clamping screw member in a lowered or engaged position;

FIG. 10 is a perspective view of a first alternate embodiment connector member of the present invention;

FIG. 11 is a side view of the connector member of FIG. 10, showing the plug and electrical source of the present invention that are capable of being coupled thereto;

FIG. 12 is a side view of the embodiment of FIG. 11, showing the electrical source connector plugged into the connector member;

FIG. 13 is an exploded view of the connector member of FIG. 12 (without the electric coupler);

FIG. 14 is a top view of a soft tissue massage therapy tool and a second alternate embodiment connector member of the present invention that employs magnetic force and a magnetic member for coupling the connector to the tool;

FIG. 15 is a side plan view of the second alternate embodiment connector of FIG. 14 shown as being magnetically coupled to a soft massage therapy tool, and wherein the electrical source and plug are coupled to the connector;

FIG. 16 is an enlarged sectional view taken along lines 16-16 of FIG. 15;

FIG. 17 is an enlarged sectional view taken along lines 17-17 of FIG. 14; and

FIG. 18 is an exploded view of the second alternate embodiment coupler member of the present invention.

FIG. 19A is a bottom view of an alternate embodiment magnetically attachable coupler of the present invention;

FIG. 19B is an end view of the connector shown in FIG. 19A;

FIG. 19C is a side view of the magnetic coupler of FIG. 19A;

FIG. 19D is a top view of the magnetic coupler of FIG. 19A;

FIG. 19E is a sectional view taken along lines 19E-19E of FIG. 19C;

FIG. 20 is a side view of a plug receptacle useable with the magnetic coupler of FIG. 19A;

FIG. 21A is a top view of an assembled magnetic coupler of the embodiment shown in FIG. 19A;

FIG. 21B is a side view of an assembled magnetic coupler of the embodiment shown in FIG. 19A;

FIG. 22A is an exploded side view of the magnetic coupler shown in FIG. 19A;

FIG. 22B is an end view of the assembled magnetic coupler shown in FIG. 19A;

FIG. 23A is a bottom view of a second alternate embodiment magnetically attachable coupler of the present invention;

FIG. 23B is an end view of the coupler shown in FIG. 23A;

FIG. 23C is a side view thereof;

FIG. 23D is a top view thereof;

FIG. 23E is a sectional view taken along lines 23E-23E of FIG. 23C;

FIG. 24 is a side view of a plug receptacle useable with the coupler of the embodiment of FIG. 23A;

FIG. 25A is a top view of an assembled coupler of the embodiment shown in FIG. 23A;

FIG. 25B is a side view thereof;

FIG. 26A is an exploded side view of the coupler of FIG. 25A;

FIG. 26B is an end view of an assembled coupler of FIG. 25A;

FIG. 27A is a top view of a lower member of an alternate embodiment clamp-based two-piece coupler, constructed generally along the lines of the embodiment shown in FIGS. 10-13;

FIG. 27B is an end view of the lower member of the embodiment shown in FIG. 27A;

FIG. 27C is a side view thereof;

FIG. 28A is a top view of an upper member of the two-piece clamp-type connector, that is designed to mate with the lower member shown in FIG. 27A;

FIG. 28B is a side view of the upper member of FIG. 27A;

FIG. 28C is an end view thereof;

FIG. 29A is a side view of a plug receptacle for use therewith;

FIG. 29B is a side view of a screw clamp for use with the screw clamp based coupler embodiment of FIG. 27A et seq.; and

FIG. 30 is an exploded view of the screw clamp based connector of the present invention, whose components are shown in FIGS. 27A-30.

### V. DETAILED DESCRIPTION OF PREFERRED EMBODIMENT

Before the present methods, implementations and systems are disclosed and described, it is to be understood that this invention is not limited to specific methods, specific components, implementation, or to particular compositions or configurations, and as such may, of course, vary. It is also to be understood that the terminology used herein is for the purpose of describing particular implementations only and is not intended to be limiting.

As used in the specification and the claims, the singular forms “a,” “an” and “the” include plural referents unless the context clearly dictates otherwise. Ranges may be expressed in ways including from “about” one particular value, and/or to “about” another particular value. When such a range is expressed, another implementation may include from the one particular value and/or to the other particular value. Similarly, when values are expressed as approximations, for example by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Similarly, “typical” or “typically” means that the subsequently described event or circumstance occurs often although it may not occur, and that the description includes instances where said event or circumstance occurs and instances where it does not.

Throughout this application, the terms “clinician” and “therapist” are used interchangeably. These terms are not intended to be limiting. Rather, they are intended to be broadly construed to include a wide variety of medical practitioners including, but not limited to physicians, athletic trainers, physical therapists, chiropractors, massage therapists and related health care professionals.

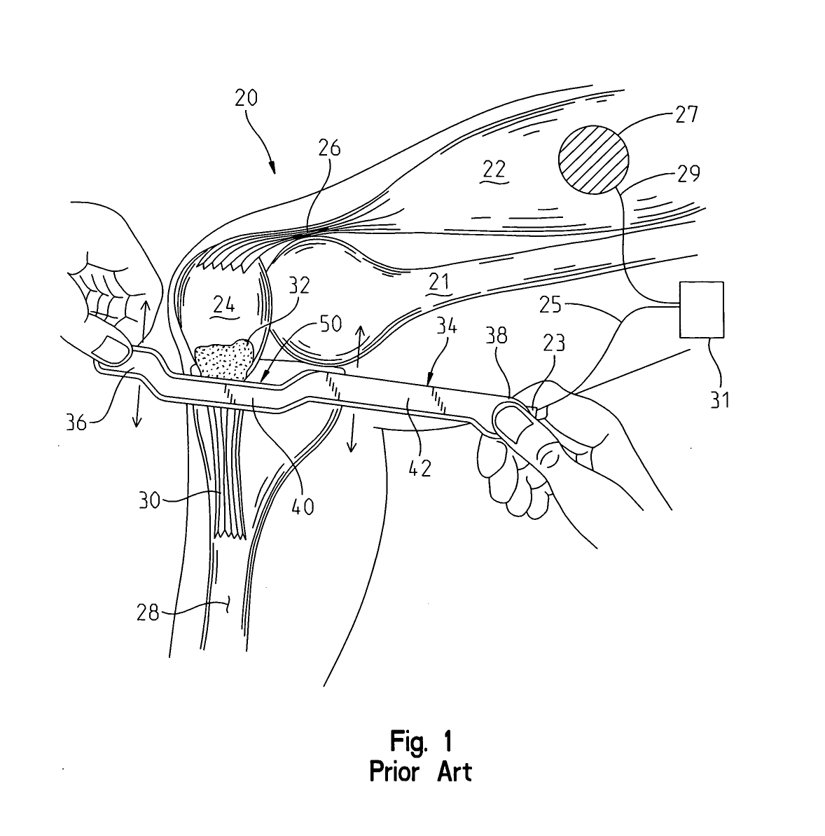

Referring now to FIG. 1, the prior art electro therapy tool of the Graston '346 and '620 patents is shown. The prior art Graston device and method will be described to help acquaint the user with anatomical, physiological and therapeutic aspects attendant to the use of the present invention. These two Graston patents, along with the others discussed above, are fully incorporated herein by reference.

In FIG. 1 there is shown a human leg 20 having a femur 21 and a quadriceps muscle 22 that is attached to patella 24 by tendon 26. Similarly, there is shown a tibia 28 that is connected to the patella 24 by a patellar ligament 30, which is subject to a great amount of stress and injury. It should be noted that other soft tissue areas in the vicinity of the knee joint have been omitted for the sake of clarity in the following description.

Once injured, scar tissue, or fibrous adhesions 32 are formed on ligament 30 as a result of the healing process. Scar tissue 32 is made up of individual fibers bound together. As scar tissue 32 comes into contact with bone, in this case patella 24, it becomes hard and reduces flexibility in the lower knee joint. The scar tissue shown in FIG. 1 is generally in a first phase. In more severe cases, scar tissue has advanced around the joint capsule and has formed on other adjacent soft tissue areas. The present invention is designed to remove or remodel scar tissue in varying degrees of advancement.

An exemplary rigid prior art tool 34 for removing scar tissue 32 from patella 24 and ligament 30 is shown in FIG. 1. Tool 34 is preferably made of stainless steel, although other suitably rigid materials such as aluminum may be utilized. Tool 34 generally comprises a handle portion and a skin-contacting portion, as well as opposite ends 36 and 38, which serve as handles. The handle portions are generally rounded to fit comfortably in the hands of the clinician. The skin-contacting portion includes “contoured” portions 40 and 42. Essentially, these portions of tool 34 are contoured to match the shape of the joint being massaged, thereby permitting greater coverage of the area to be massaged.

Tool 34 includes an outer peripheral edge that varies along the circumference of the tool. One portion of the peripheral edge is a flat edge 44 which is generally a flat surface that is perpendicular to the top 46 and bottom 48 surfaces of tool 34. A second portion of the peripheral edge is a so-called “beveled” edge 50 and includes an upper beveled surface and a lower beveled surface. The upper and lower beveled surfaces may be beveled at various angles with respect to top and bottom surfaces, respectively. As an example the upper and lower surfaces can be beveled at about 135° with respect to the top and bottom surfaces, respectively. The top and bottom surfaces meet at an edge.

Tool 34 is just one of a plurality of tools that may be provided as a set for use in soft tissue therapy according to the present invention, and other tools are shown in the above referenced Graston patents. It is noted that the tools illustrated herein (and in the Graston patents) do not comprise an exhaustive list of tools that may be required for treatment of all soft tissue areas used, but are merely illustrative of the different shapes and sizes of tools that may be utilized to treat some parts of the body according to the present invention. In addition, it is noted that each of the tools shown herein may be used on different parts of the body as needed.

As further shown in FIG. 1, positive electrode, or cathode, 23 is electrically connected to tool 34. Although shown only with regard to tool 34 in FIG. 1, similar electrode connections may be formed in any of the other tools of the set. Negative electrode, or anode, 27 shown in this instance in the form of a circular pad, is shown in FIG. 1 positioned in the vicinity of the “belly” of quadriceps muscle 22. Lead cord 25 extending from cathode 23 and lead cord 29 extending from anode 27 are attached to source 31 of electrical current. Source 31 is shown schematically in FIG. 1.

Source 31 may be any source of current used in conventional electrical stimulation therapy (“electro therapy”). Preferably, source 31 is capable of generating the various waveforms commonly utilized in electro therapy, and is capable of providing varied pulse rates. One such device that is appropriate for use in the present invention is the Rich-Mar Theratouch 4.7, available from Rich-Mar Corporation of Inola, Okla. Cathode 23 and anode 27 are also available from Rich-Mar. Preferably, anode 27 comprises a conventional circular padded electrode, however other electrodes presently used for this purpose are also acceptable. Anode 27 may be adhered to muscle 22 by any method known in the art, such as by a strap.

In order to perform soft tissue massage according to the present invention, a gel, such as ALOE-SOUND GEL PLUS, also available from Rich-Mar, is first applied to the affected soft tissue area and to the area to be covered by the anode. As used herein, the term “soft tissue” generally refers to a muscle, ligament, tendon, or any combination thereof. It should be understood that the system of the present invention may be used on any part of the body in which a soft tissue injury has occurred and scar tissue has been built up as a result of the healing process. The particular soft tissue area illustrated herein, a knee joint, is merely illustrative of one possible application of the present invention.

Once the affected soft tissue area has been properly lubricated, an appropriate tool such as tool 34 is selected. Cathode 23, electrically connected to source 31, is plugged into the receptacle at end 38 of tool 34. Anode 27, also electrically connected to source 31, is positioned at the belly of muscle 22, in a manner well known in the art of electro therapy. The appropriate waveform and pulse is then selected by the therapist, and the appropriate controls on source 31 are adjusted to provide the desired output. For edema reduction, high voltage pulsed galvanic (HVPG) current is preferred.

Tool 34 is then passed across the knee joint in the direction of the arrows as shown in FIG. 1 so that the precise location of scar tissue 32 is determined. Generally, “bumps” may be felt through the tool to indicate the presence of scar tissue at a particular location on the soft tissue. Such scar tissue is often undetectable by merely using one's hands alone. The applicants have found that a beveled edge is very useful for locating hardened scar tissue or tissue close to bone. In contrast, the blade edge is useful for locating scar tissue that is not in such a hardened state.

Once the location of the scar tissue is determined, the scar tissue should be broken up. This is accomplished by moving the tool 34 in the manner shown in FIG. 1, so that beveled edge 50 breaks up hardened scar tissue 32. Once tissue 32 begins to break up, an irritation occurs which causes swelling of the scar tissue. The electrical stimulation provided to the patient through tool 34 acts upon and reduces this irritation.

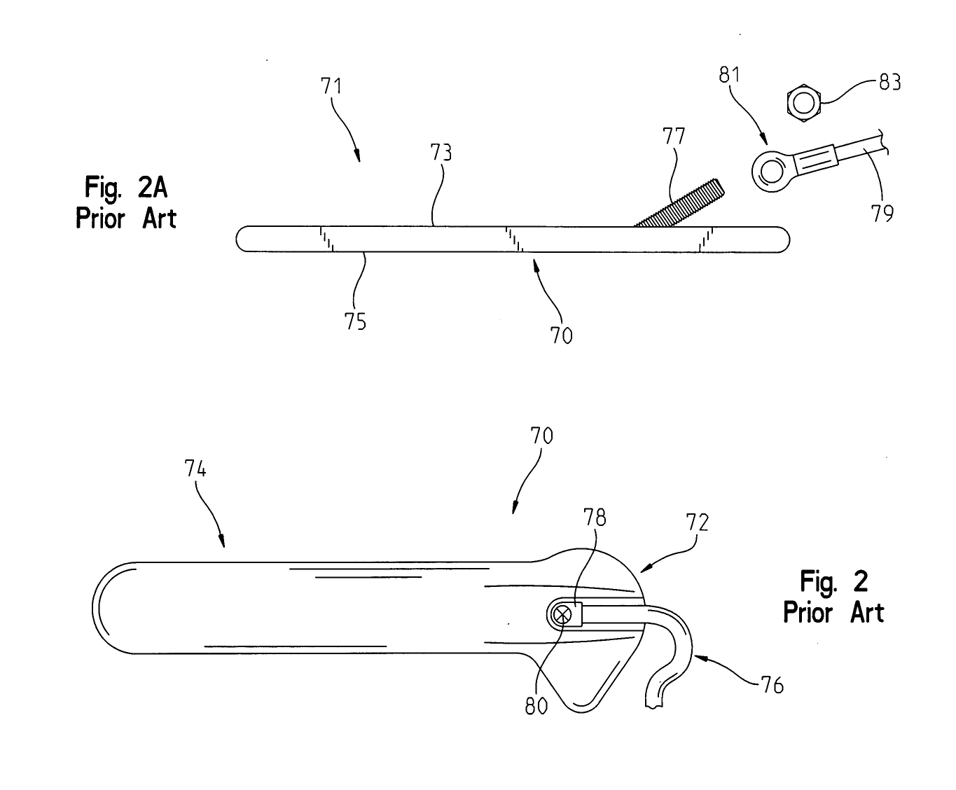

Turning now to FIG. 2, a prior art electro therapy tool 70 is shown. Electro therapy tool 70 is generally tomahawk-shaped and is made out of a rigid material, and preferably, a rigidly electro conductive material, such as stainless steel. The tomahawk-shaped therapy tool 70 has a head end portion 72 that is shaped similarly to the head of a tomahawk and a tail portion 74 that is generally blade-shaped, much like a thickened tongue depressor. An electrical source wire 76 includes an end connector 78 that is connected via a screw 80 to the head portion 72 of the tomahawk-shaped tool. Through this connection, current that travels through wire 76 can be electrically coupled to, and pass into the tomahawk-shaped soft massage electro therapy tool 70, so that the electricity flowing through the tool can ultimately flow into the soft tissue and/or scar tissue adhesions of the patient to provide appropriate electro therapy thereto as discussed above.

One difficulty with the prior art tool is that the connector 78 is generally permanently connected to the tool 70 or is at least difficult and time consuming to remove. As such, one usually employs a separate tool and wire combination, for each of the varieties of tools. Additionally, if one did not wish to be in a position where one was removing and adding wires on a regular basis, it was generally more convenient for the user to buy two sets of tools. The first set of tools would be a standard, non-electro therapy set, and the second set would be an electro therapy set.

FIG. 2A is a side, exploded view of another prior art electro therapy soft tissue massage tool 71. Tool 71 has a top side 73 and a bottom side 75. A post, here shown as a threaded post 77 is fixedly mechanically and electrically coupled to the top side 73. A current source, her shown as wire 79 has a washer like terminus 81 that has a central aperture that is sized to receive the treaded post 77. A fastener, here shown as a locking bolt 83 has an aperture containing female threads for receiving the male threads of the post 77.

Although the tools perform their function in a highly workmanlike manner, room for improvement exists as the tools can be made more convenient to use; and possibly more cost-effective by enabling the tools to do double-duty as both electro therapy tools, and also as conventional, non-electro therapy soft tissue massage tools.

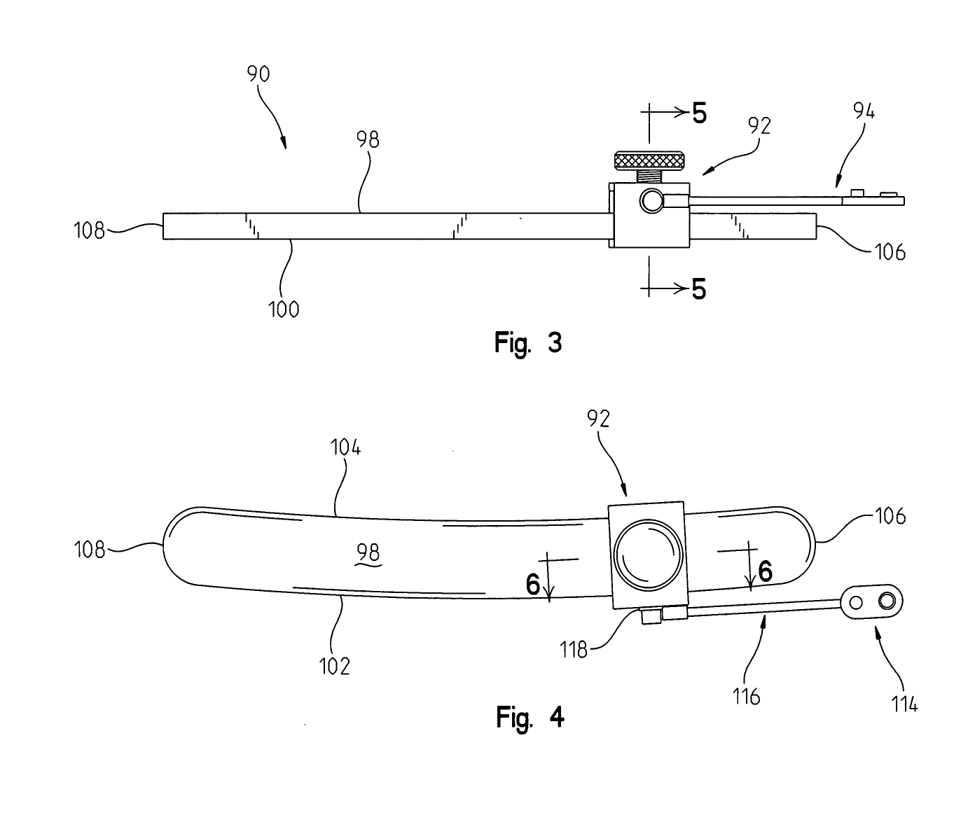

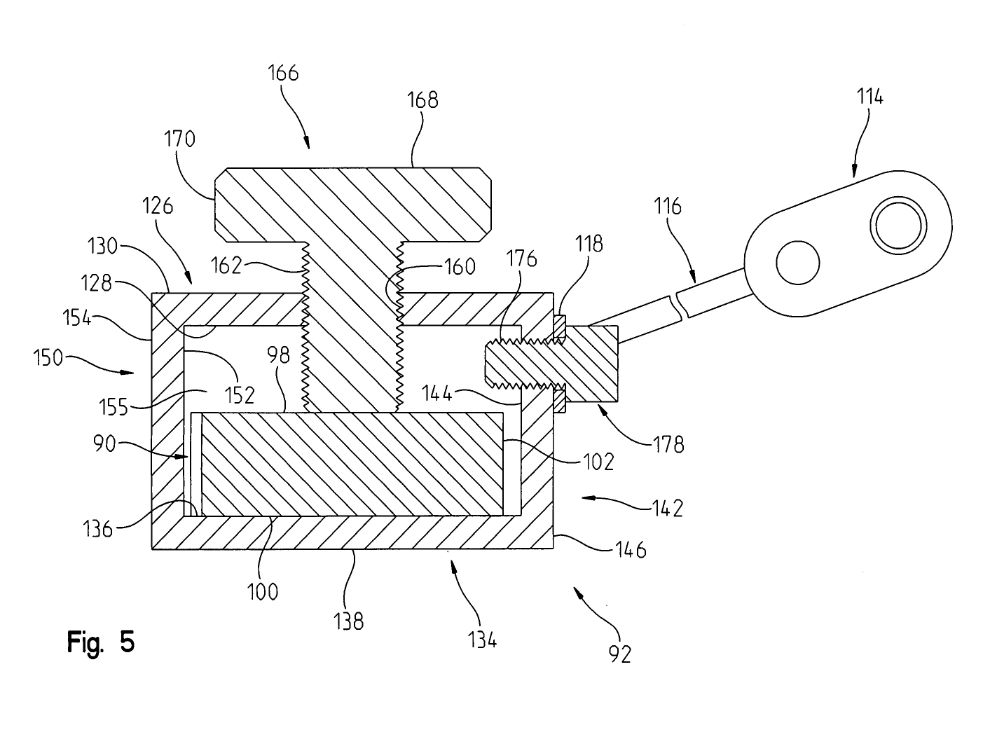

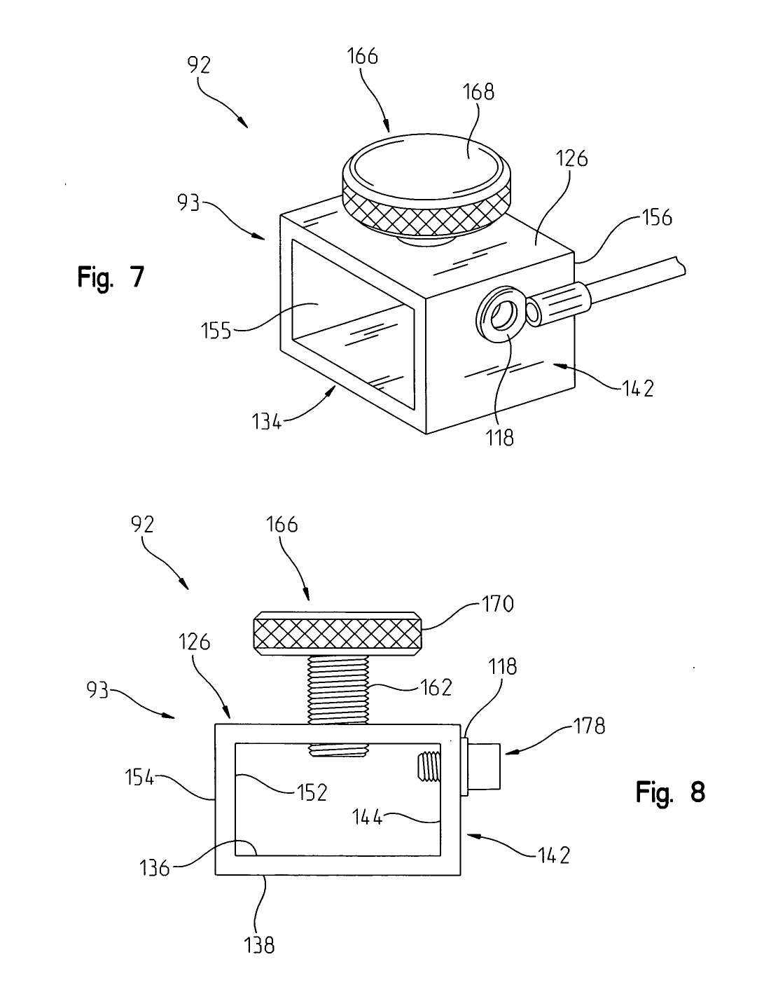

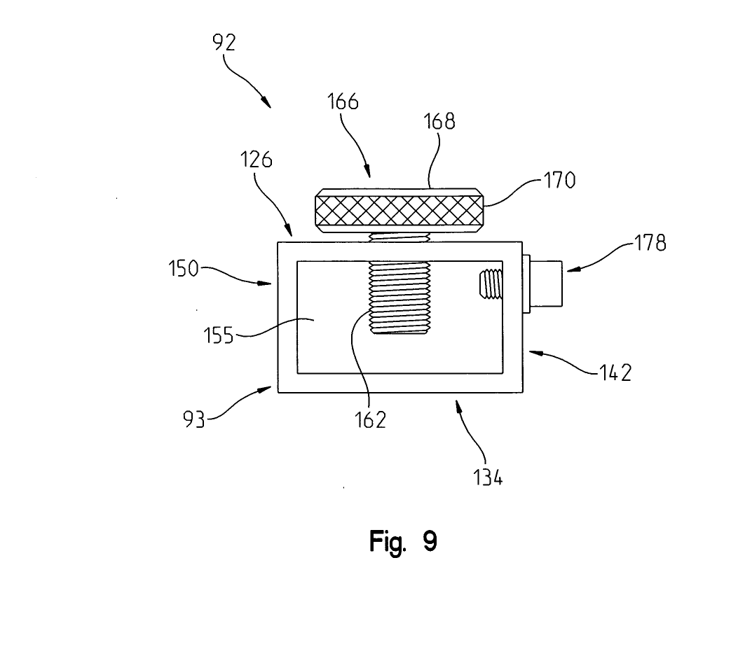

A therapy tool system and a connector 92 of the present invention is shown in FIGS. 3-9. The therapy tool system comprises a rigid therapy tool 90 for passing across a patient's skin for loosening subcutaneous tissue. The tool 90 includes an electrically conductive portion 50 (FIG. 1) engageable with a patient's skin. A current source 114 is provided for providing an electric current. A connector member 92 is provided that is selectively attachable and removable from the tool 90. The tool member 90 is coupled to the current source 114 for conducting current between the current source 114 and the tool 90. The rigid tool 90 includes a conductive path for conducting current from the connector 92 to the electrically conductive portion of the tool 50, to enable current delivered to the connector 92 from the current source 114 to be conducted to the electrically conductive portion of the tool 50 and transferred from the tool 90 through the patient's skin to subcutaneous patient tissues.

Preferably, the therapy tool connector member 92 includes a sleeve portion 155 for receiving the rigid tool 90, and a clamp 166 for fixedly removably coupling the connector 92 to the tool member 90, wherein the subcutaneous tissue comprises scar tissue underlying patient soft tissue, all of which is disposed subcutaneously.

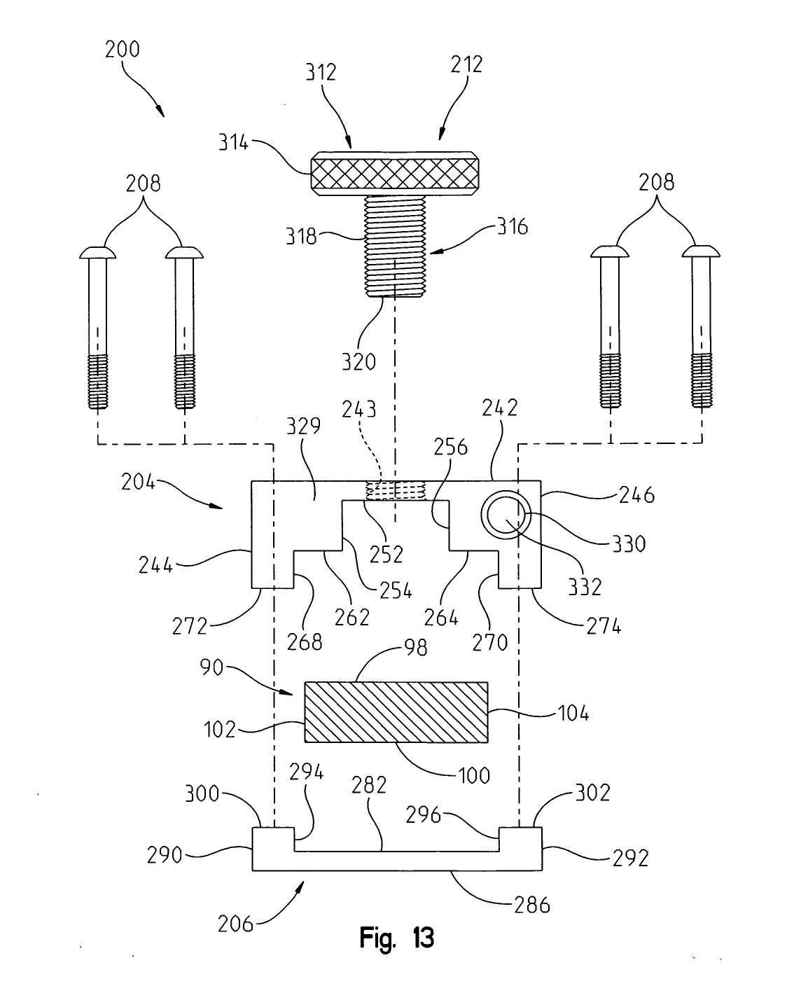

In a preferred embodiment, the sleeve portion 215 (FIG. 11) can include a relatively wide portion 218 for receiving the rigid tool 90, and a relatively narrower portion 216. The relatively narrower portion 216 is partially defined by shoulders 262, 264 (FIG. 13) for limiting the movement of the tool 90 received in the relatively wider portion 216.

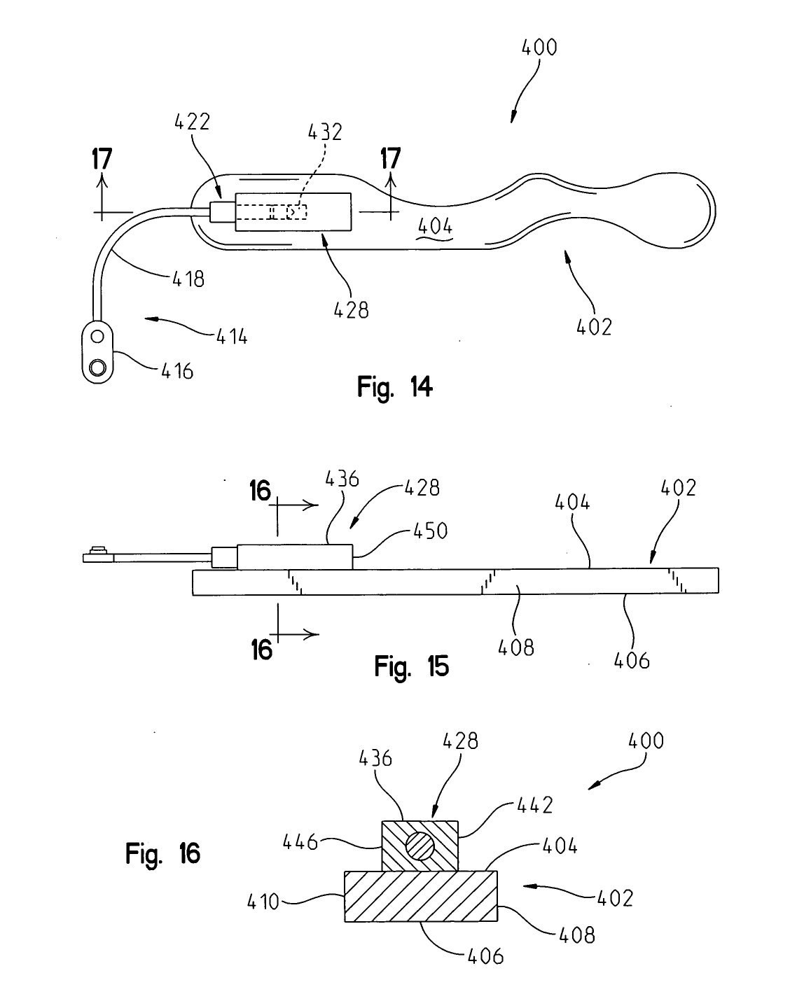

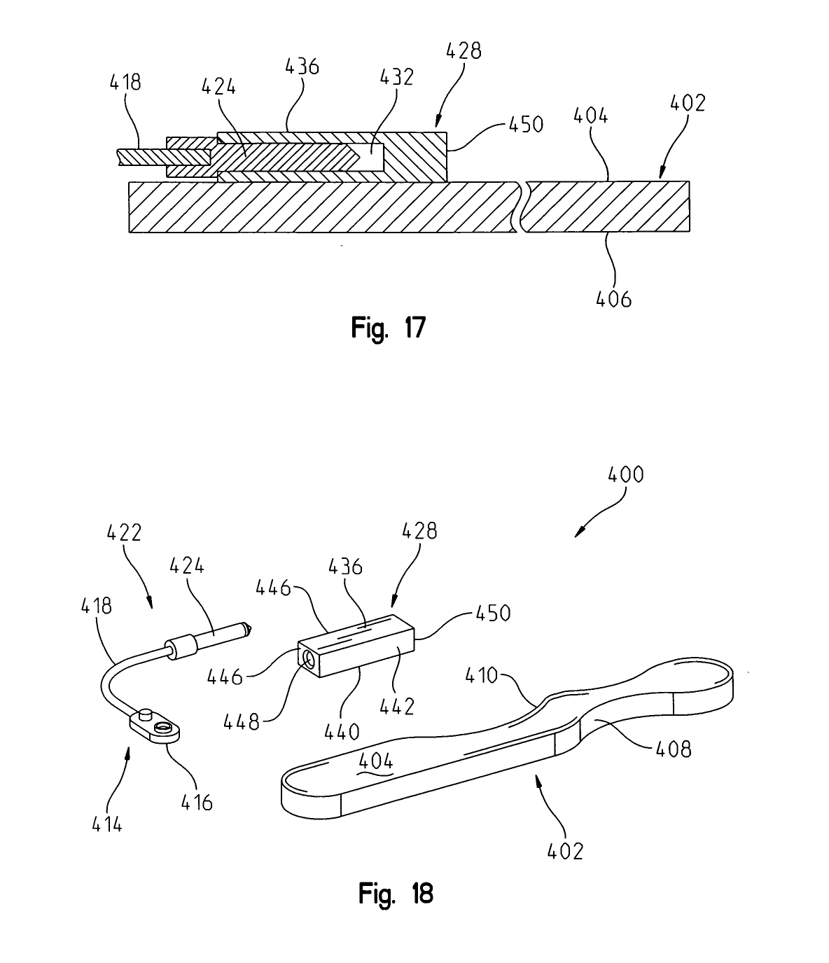

In another preferred embodiment, the connector member 428 comprises a magnetic connector member 428 (FIGS. 14-18) that is capable of being magnetically coupled and electrically coupled to the rigid tool 90. The magnetic connector member 428 can include a plug receptacle 432 for receiving a plug 424. The current source 416 can include a plug member 424 capable of being coupled to the plug receptacle 432.

Returning back to FIGS. 3-9, the tool 90 is generally blade-like in configuration, and has a somewhat planar upper surface 98 in a somewhat planar lower surface. The particular tool shown in FIGS. 3 and 4 includes a convex first side edge surface 102, and a concave second side edge surface 74. The tool 90 includes a first end edge 106 that is disposed at the first end of the tool 90, and a second edge 108 is disposed at a second end of the tool 90.

The electrical power source system 94 includes a power source 114 that can be a source of either AC or DC current, and can be provided by a “plug-in source” to employ AC current, or a battery back. Additionally, if DC power is required, a transformer can be provided to transform AC power to DC power. Examples of power sources and controls therefor are discussed above. The wire source is coupled to a wire 116 having a proximal end at the power source 114, and a distal end at a connector end piece 118. The connector end piece 118 can be a screw-receiving ring, such as is shown in FIGS. 3-9, or else a plug-type member (See FIG. 11). The purpose of the end piece is to conduct the electrical current from the wire 116 to the tool 90.

Preferably, the connector 92, along with tool 90 are both electrically conductive, so that current passing through the connector 92 can pass into the tool 90, and hence, into the patient through contact between the current-passing tool and the patient's tissue when the patient is being massaged with the tool 90. The connector shown in FIGS. 3-9 is a generally sleeve-like connector that includes a generally rectangular top plate having an interior surface 128, and an exterior surface 130. The sleeve-like connector 92 also includes a bottom plate 134 having an interior surface 136 and an exterior surface 138. Preferably, the top and bottom plates 126, 134 are generally identical in size and configuration wherein the top and bottom plates 126, 134 are disposed in parallel planes.

A front face plate 142 extends between the top and bottom plates 126, 134 and includes an interior surface 144 and an exterior surface. A rear plate 150 is disposed in a plane generally parallel to the front plate, and generally perpendicular to the top and bottom plates 126, 134 and itself also includes an interior surface 152 and an exterior surface 154.

The respective interior surfaces 128, 136, 144, and 154 define a hollow interior of the connector 92, that comprises the sleeve 155 that is sized and configured for receiving the tool 90 therein. It will be noted that the connector includes a first open end 155, and a second open end 156, that are placed in an opposed relation. The tool 90 is designed to pass through these open ends 155, 156 so that it may reside within the interior of the connector.

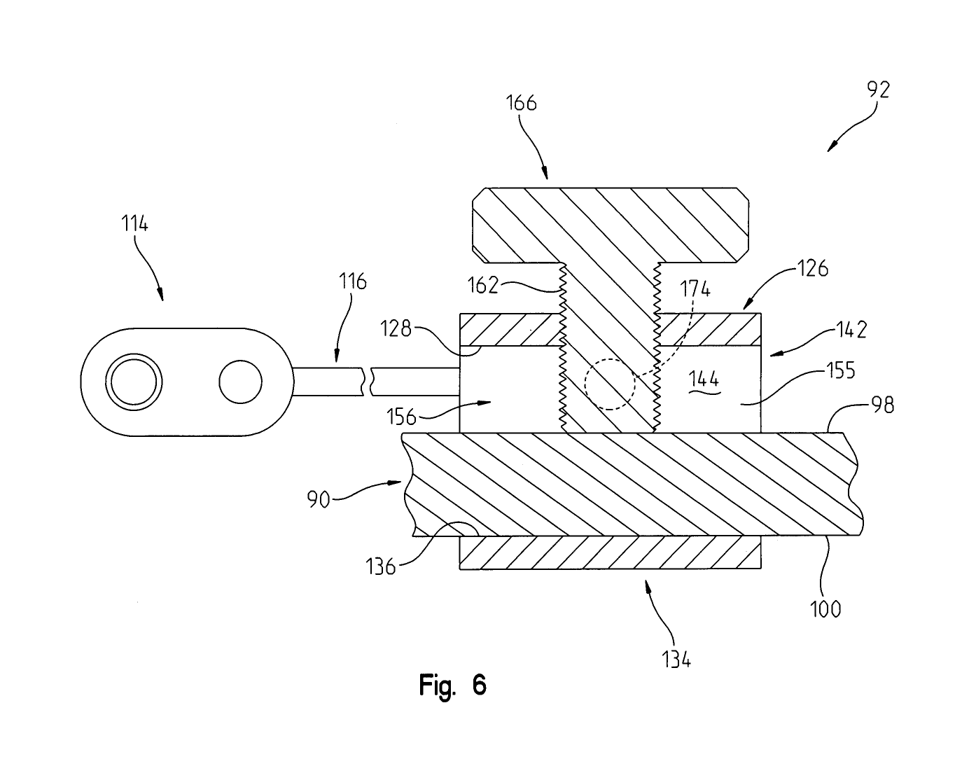

As best shown in FIG. 6, the bottom surface 100 of the tool 90 is placed against the interior surface 136 of the bottom plate 134. Preferably, the bottom surface 100 of the tool 90 is placed in electrical contact with the upper surface 136 of the bottom plate 134, so that electrical current that is conducted in the connector 192 can be transferred from the connector, through the bottom surface 136 into the tool 90, so that electricity can flow through the tool 90, and into the tissue of the patient whose tissue is being massaged by the tool.

The top plate member 126 of the connector includes a threaded central aperture 160 that is sized and configured for receiving a threaded male stud portion 162 of a clamping screw 166. Clamping screw 166 has a disc-shaped head 168 that includes a knurled edge portion 170 for facilitating hand loosening and tightening of the clamping screw 166. The threaded clamping screw 166 can be threaded to cause the stud portion 162 to move axially, to a point wherein the distal end of the threaded stud portion 162 engages the upper surface 98 of the tool.

When the threaded clamping screw 166 is sufficiently tightened, the engagement between the clamping screw 166 and the tool 90 is capable of fixedly positioning the connector 92, onto the tool 90. Additionally, the threaded tight engagement of the clamping screw 166 against the upper surface 98 of the tool 90, presses the bottom surface 100 of the tool into intimate engagement with the upper surface 136 of the bottom plate 134 so that good electrical contact can be made between the tool 90 and the bottom plate 134 of the connector 192 to facilitate the passage of current between the connector 192 and the tool 90.

The front plate 142 is shown in FIG. 6 as including an aperture 174. Threaded aperture 174 is provided for receiving a threaded stud portion 176 of a threaded wire connector screw 178, that preferably passes through the center of ring 118, to fixedly couple the wire 116 to the connector 92, so that current passed through the wire 116 can pass into and through the connector 92, and ultimately, find its way into the tool 90, and thence, into the tissue of the patient being massaged with the tool 90.

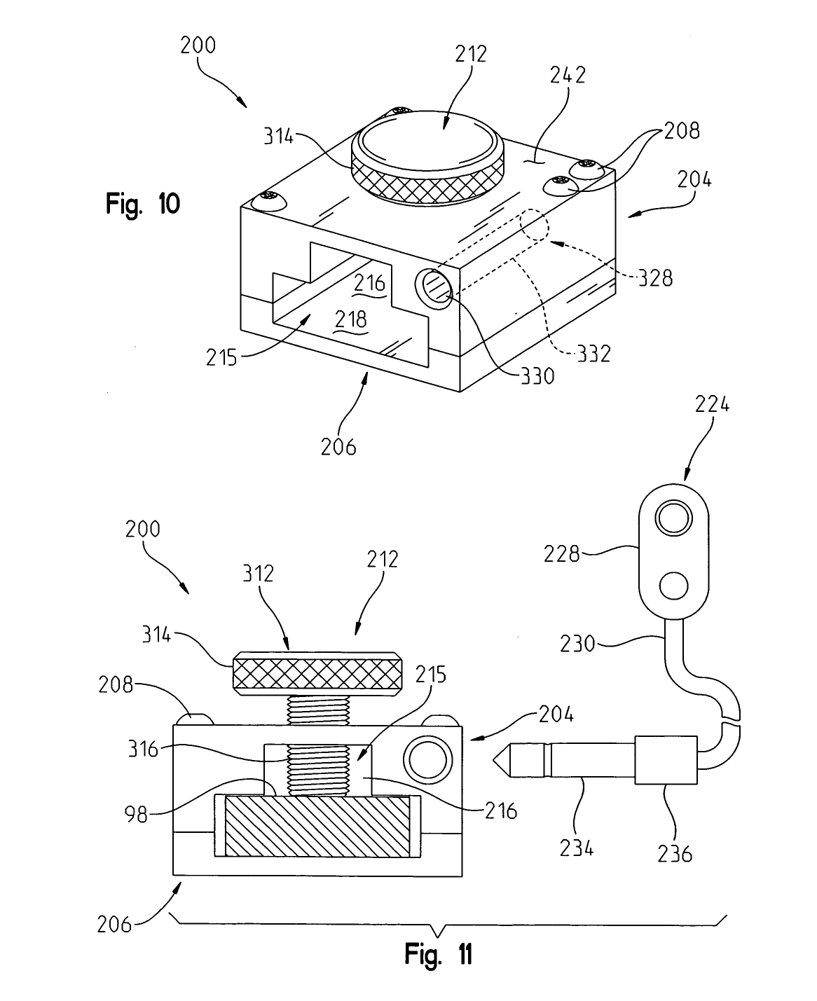

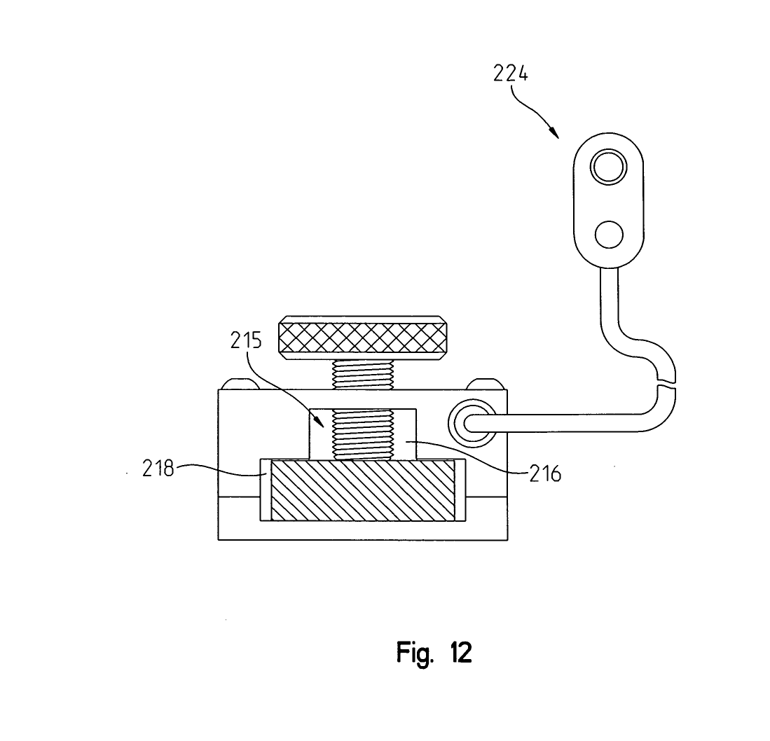

A first alternate embodiment connector member 200 is shown in FIGS. 10-13. At the present time, the device shown in FIGS. 10-13 is believed by Applicants to be a version that is more preferred than the version shown in FIGS. 1-9.

The second alternate embodiment connector 200 is a two-piece connector that includes an upper portion 204, and a lower portion 206. The upper and lower portions 204, 206 are sized and configured for being joined together into a single unit. Preferably, the connector 200 is formed as a unitarily formed device. Alternately, the upper and lower portions 204, 206 can be formed as two separate members 204, 206 that are coupled together by fasteners such as machine screw connector 208. A plurality of screw like connectors 208 can be provided that are insertable into apertures that are formed in the upper and lower portions 204, 206, and through which the screw-like connectors 208 can pass, for connecting together the upper and lower member 204, 206. An example of such aperture placement is shown in FIG. 11.

Similar to the embodiment shown in FIG. 1, the second embodiment connector 200 also includes a screw type clamping screw 212 for fixedly coupling the tool 90 in a desired position within an interior passageway 218 of the connector 200, and fixedly positioning the tool 90 in place. Additionally, the clamping screw connector 212 helps to hold the lower surface 100 of the tool 90 in a closely engaged and contacting position with an upper interior surface 282 of the lower member 206 to provide a good electrical connection between the tool 90, and the connector 200.

As best shown in FIG. 10, the connector 200 includes an interior passageway that extends between a first open side member 304 and a second open side member (not shown). The interior passageway 114 includes a relatively reduced width portion 216, and a relatively enlarged width portion 218. Enlarged width portion 218 is provided for being wide enough to accept the full width of the tool 90. Additionally, the shoulders 262, 264 that are formed by the first and second horizontally extending shoulder members help to maintain the tool 290 within the enlarged diameter portion 218, that helps to provide both vertical stability, and horizontal stability, along with helping to reduce the likelihood of the tool 90 being able to twist, and rotate about either its longitudinal axis or its lateral axis.

An electrical service provider 224 is provided for providing electrical current to the connector 200, so that the electrical current so provided can be passed through the connector 200, and into the tool 90, and ultimately, into the tissue of a patient upon whom the tool is being used. The electrical service provider 224 includes a power or current source 228 of the type described above. A wire 230 conducts current away from the power source 228 and terminates in a plug member 232.

Plug member 232 includes an insertable male plug end 234 that is sized and configured to be inserted within the female plug receiving aperture 330 and passageway 332. The plug member 232 also includes a non-conductive grip 236 that preferably has an enlarged diameter relative to the diameter of the insertable plug end 234, to facilitate the user inserting and removing the plug in 234 into and out of the interior passageway 332.

The upper member 204 is best shown in FIG. 13 as including a horizontally disposed top exterior surface 242. The top exterior surface 242 includes a threaded passageway 243 that extends generally perpendicular to the plane of surface 242 and extends through the upper member 204. The passageway 243 is threaded, sized and configured for receiving the stud portion 316 of the threaded clamping screw 212.

The upper portion member 204 also includes a front facing exterior surface 244, and a rear facing exterior surface 246. The front and rear exterior facing surfaces 244, 246 terminate at their lower ends in horizontally disposed distal end surfaces 272, 274 that are sized and positioned for engaging upper end surfaces 300, 302 of the lower member 206.

The upper member 204 also includes a first, horizontally disposed upper interior facing surface 252, along with first and second vertically disposed side surfaces 254, 256. The horizontally disposed surface 252, and first and second side surfaces 254, 256 generally define the upper, reduced width portion 266 of the interior passageway of the connector.

The first and second horizontally disposed shoulder surfaces 262, 264 extend horizontally and radially outwardly relative to the axially extending passageway 216 from the first and second vertical surfaces 254, 256, and terminate at the radially outwardly most portions in first and second distal side surfaces 268, 270 respectively.

As discussed above, the shoulders 262, 264 define the upper edge of the enlarged diameter portion 218 and are provided for helping to limit movement of the tool 90, when the tool 90 is placed in the lower, enlarged width portion 218 of the connector. The first and second horizontally disposed distal end surfaces 272, 274 extend between the first and second distal side surfaces 268, 270, and the first and second vertically disposed exterior side surfaces 244, 246, respectively.

The lower member 206 includes a horizontally disposed generally planar interior tool engaging surface 282, that is sized and positioned for receiving a major surface of the tool 90 such as underside surface 100, and engaging the underside surface 100 of the tool 90 in an electrically conductive relationship. The lower member 206 also includes a generally planar horizontally disposed exterior surface that is disposed in a plane generally parallel to the horizontally disposed interior surface 282.

First and second generally vertically disposed exterior surfaces 290, 292 are provided along with first and second, vertically disposed interior surfaces 294, 296 that are placed in a generally opposed, and parallel plane relationship with the vertically disposed exterior surfaces 290, 292. First and second generally horizontally disposed end surfaces 300, 302 are provided to extend between the vertically extending exterior surfaces 290, 292 and the vertically extending interior surfaces 294, 296 respectively. The end surfaces 300, 302 are sized, positioned and configured to mate with the end surfaces 272, 274 of the upper member 204, such that the two surfaces create an electrically conductive relationship between the upper member 204 and the lower member 206.

The clamping screw 212 is similar in size and configuration to the clamping screw 166 shown in FIGS. 3-9. The clamping screw 212 includes a disc shaped head 312 having a knurled outer surface 314 to improve the user's grip on the head 312, to thereby make it easier for the user to rotate the clamping screw 212 into and out of engagement with the tool 90. The clamp 212 also includes an axially extending stud member 319 having an upper end that is coupled to the underside surface of the disc-shaped head 312 and a generally planar lower surface 320. A series of male threads 318 are formed on the axially extending stud 316.

The generally planar end member 320 is provided for engaging the upper surface 98 of the tool 90 to create an electrical contact between the clamp 212 and the tool 90, if such an electrical connection is necessary. However, since it is not necessarily required that there be an electrical connection between the clamp 212 and the tool 90, the screw clamp 212 can be made from a non-conductive material, such as a heavy duty plastic, if so desired, without necessarily adversely affecting the performance of the device.

Optionally, a gripping member, such as an enlarged diameter foot having axially extending teeth (not shown) can be coupled to the distal end of the stand 316 to provide an enlarged or more secure gripping mechanism for the stool 316 of the clamping screw 212 to engage the tool. As another alternative, an insulating foot (not shown) can be coupled to the distal end of the stud 316, to provide either or both of an electrical insulation or vibrational insulation between the distal end of the stud 316 of the clamping screw 212 and the tool 90.

The female plug receptacle 328 has an opening 330, that is disposed on a generally vertically disposed side surface 329 of the upper member 204. The opening 330 opens into an interior blind hole type passageway 332 that is sized to snuggly receive the insertable cylindrical male plug 234, so that a good electrical connection can be made between the male plug 234 and the interiorly facing surfaces that define the interior blind hole type passageway 332.

A variety of processes exist by which the second connector 200 can be produced. One method would be to separately extrude the upper and lower portions 204, 206. The passageway 332 for the female plug receptacle, along with the passageway for receiving the screw connectors 208, can then be machined into the upper and lower members after extrusion. Additionally, the vertical screw clamp 212 receiving passageway 243 can also be formed by a machining process.

After the parts are formed, the upper and lower members 204, 206 can then be joined by mating the upper 204 and lower 206 members together at their respective end portion surfaces 272, 274, 300, 302 so that the apertures for the screw connectors 208 are aligned in the upper and lower members 204, 206. The screw connectors 208 are then inserted in their apertures, and threadedly engaging the lower ends of the screw connectors 208 with female threaded passageways that form the screw connector receiver in the lower member 206.

The second alternate embodiment connector system 400 is shown in FIGS. 14-18. Connector system 400 is designed for coupling an electrical service source to the soft tissue massage therapy tool 402, so that electricity can be directed through the tool 402 and into the tissue of the patient upon whom therapy is being performed with the tool 402. The massage therapy tool 402 is a variant of the tool 90 shown in other drawings, and may be identical to, or selected from one of the tools shown in the earlier Graston patents discussed above. The tool 402 is generally tongue depressor-shaped, insofar as it is thin and blade-like, and includes an upper surface 404, a lower surface 406, a first side surface 408 and a second side surface 410.

An electrical service assembly 414 is provided for providing electricity to the connector member 428. The electrical service assembly 414 includes a power source 416 of the type described above. A conductor wire 418 is provided for conducting electrical current from the power source 416 to a plug 422, that couples the electrical service 414 to the connecting member 428. As shown in connection with the embodiment shown in FIG. 13, the plug 422 includes a cylindrical male member 424 having a frusto-conical tip that is insertable into a passageway 432 of the connector member 428. A variety of different plugs can be used, depending upon the desires of the user, and taking into account factors such as durability, cost, size and current to be delivered.

The connector member 428 comprises a block of magnetizable electrical conducting material, such as stainless steel. The connector member 428 is coupled to the upper surface 404 of the tool 402 through the magnetic attraction of the tool 402 with the magnetizable block that comprises the connector member 428.

The connector member 428 includes an axially extending passageway that is sized and configured for serving as a female receptacle for receiving an insertable male plug member 424. Preferably, the passageway 432 is capable of conducting electrical current, so that current that is being provided through the plug 424 will travel into a connector member 428, and, through the engagement of the lower surface 440 of the connector member 428 to the upper surface 404 of tool 402, will cause the current to flow through the tool 402, and ultimately into the tissue of the patient upon whom the tool is being used.

The connector member 428 is generally rectangularly cuboid in shape and includes a planar upper surface 434 and a planar lower surface 440 that are disposed in spaced, parallel planes. The connector member 428 also includes a front surface 442 and a rear surface 444 that are disposed both in planes generally perpendicular to the planes of the upper and lower surface 438 and 440, although the front surface 442 and rear surface 444 are disposed in planes that are generally parallel to each other.

The connector member 428 further includes a first side member 448 that includes a first side surface 446 that includes an opening 448 of the passageway 432, and through which the male plug member 424 is inserted, so that the male member 424 can reside within the female passageway 432. Finally, the connector member 428 includes a second side surface 450 that is disposed in a plane generally parallel to the plane of the first side surface 446, and perpendicular to the planes of the upper, lower, front and rear surfaces 438-444.

The magnetic connector member 428 operates generally similarly to the connector members discussed above, except that there is no requirement for a mechanical type clamping connection between the connector 428 and the tool 402. Rather, the magnetic attraction between the connector member 428 and the tool 402 secures the two to each other. This magnetic attraction makes it very easy and simple to connect the source of electricity 414 to the tool 402, and disconnect it.

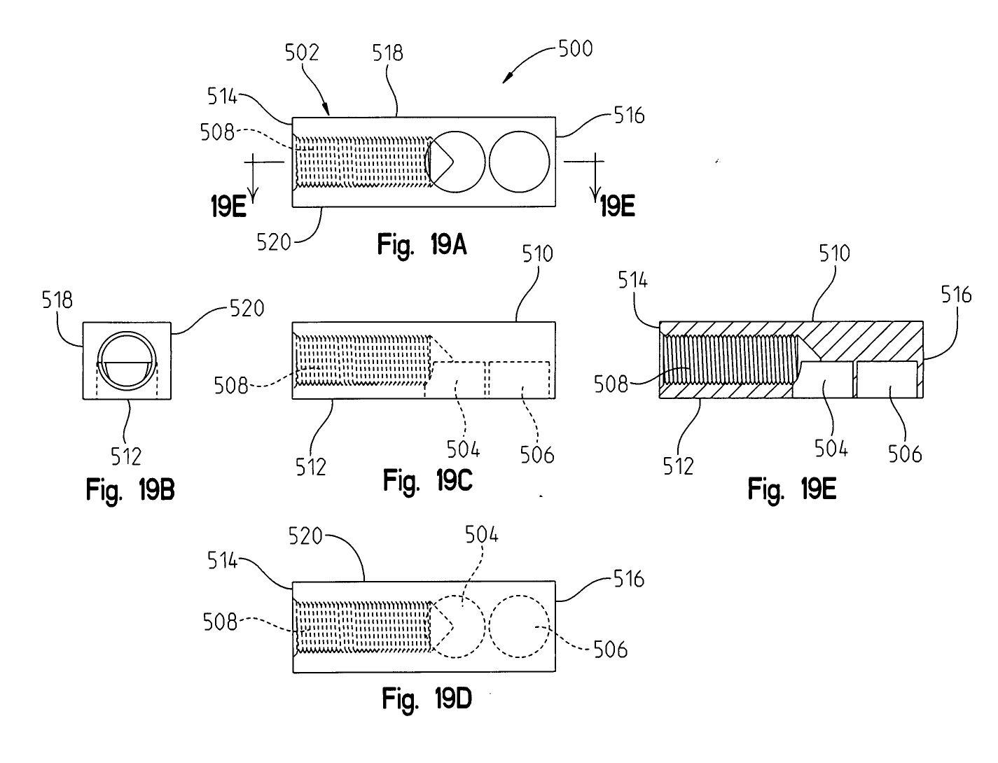

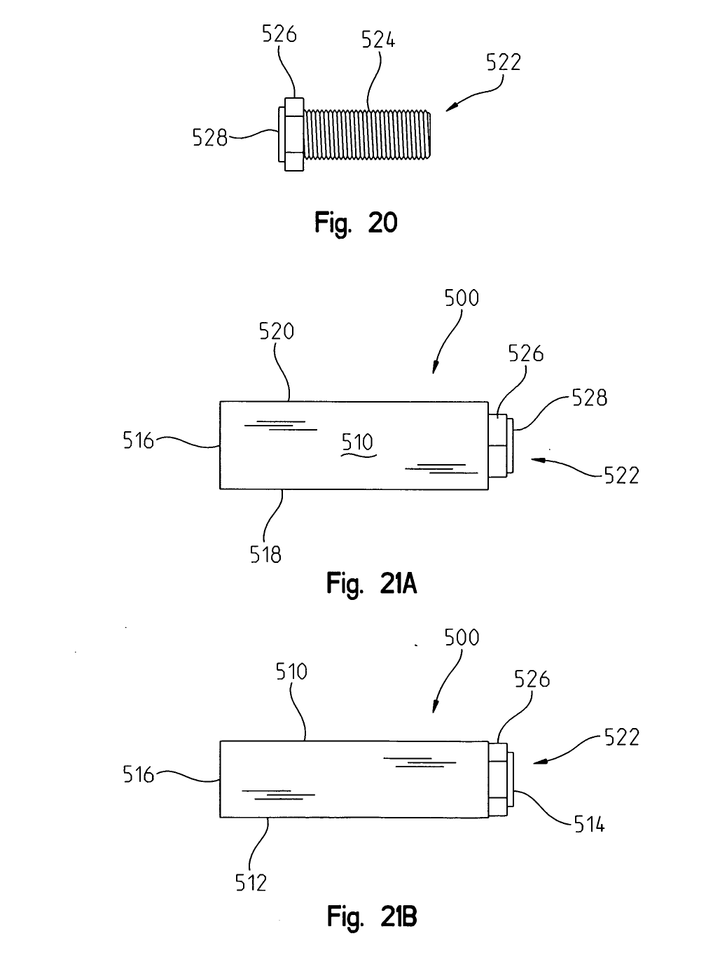

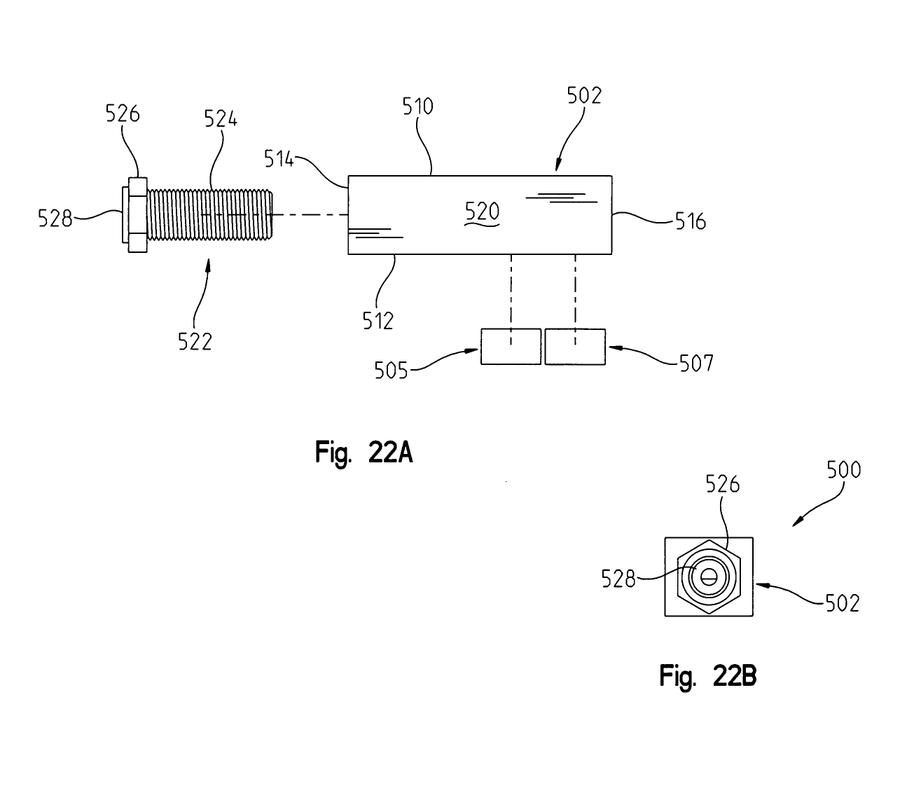

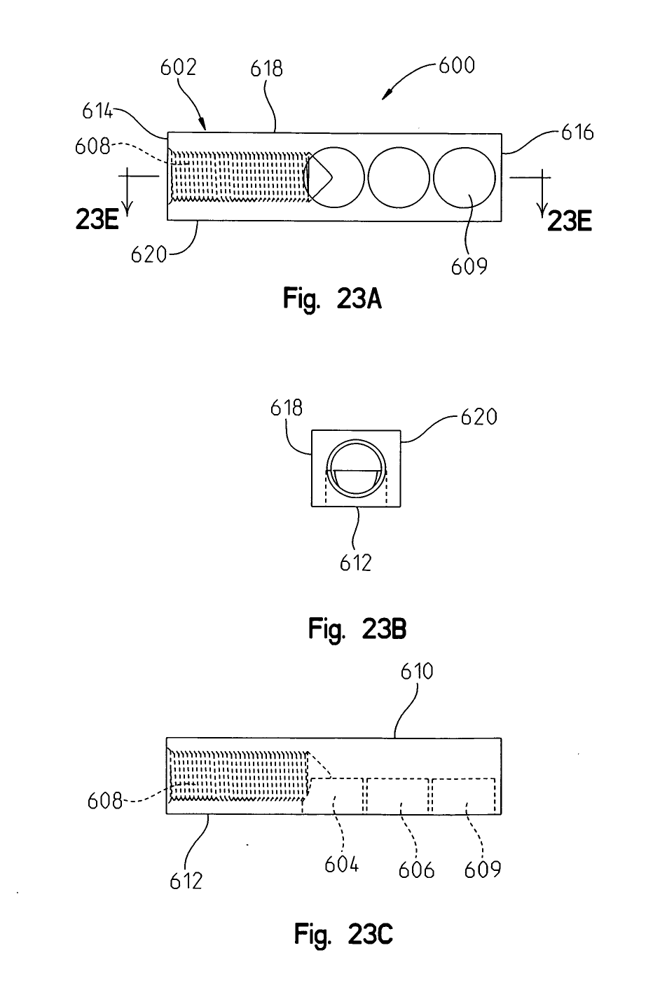

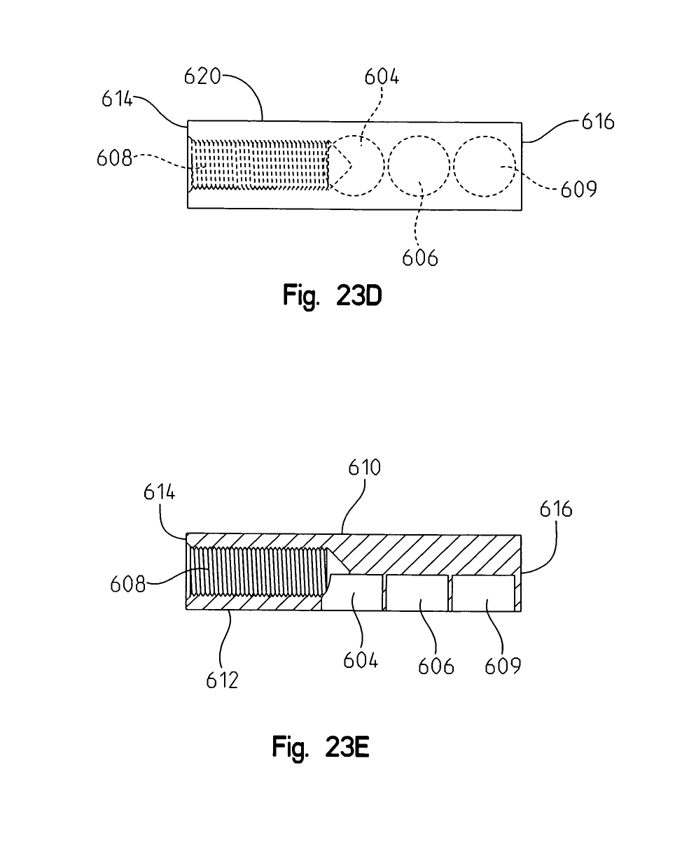

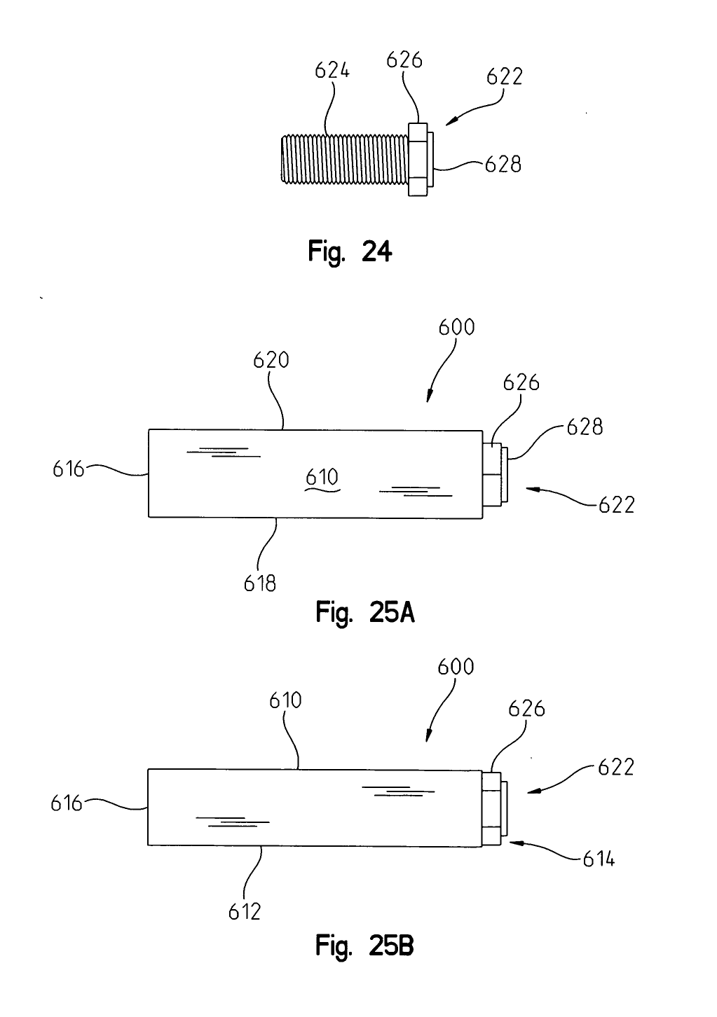

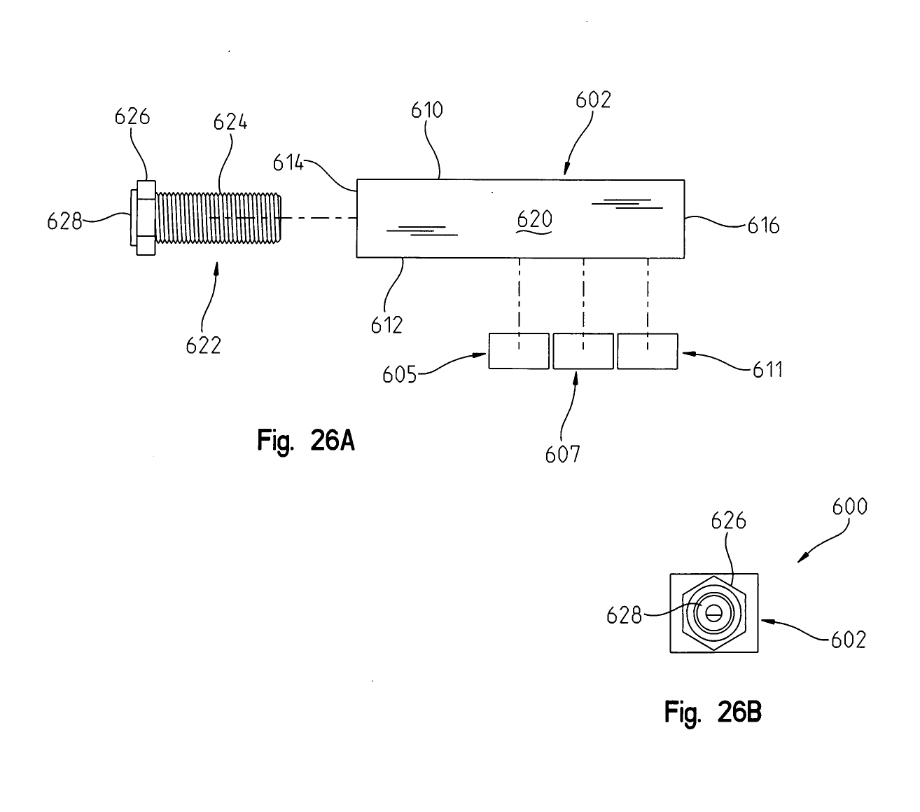

Turning now to FIGS. 19A-22B, an alternate embodiment magnetically attachable coupler 500 is shown for use in connection with a therapy tool. The magnetically attachable coupler 500 is, from a functional standpoint, a variant of the embodiment shown in FIGS. 14-18, and operates in a generally similar manner.

In particular, an electrical source (not shown) is provided that contains a plug (not shown), that is insertable into a plug receptacle 522 of the coupler 500 for providing a source of current to the coupler 500. The current so provided is then transferred to a therapy tool (e.g. tool 90) to which the magnetically attachable coupler 500 is attached. This current is then transferred, through a conductive path of the tool 90, to the patient's skin, and ultimately to subcutaneous tissue, such as scar tissue adhesions, upon which the current can act in a therapeutic manner.

The coupler 500 includes a housing 502 that is preferably made from a current conducting, magnetizable material such as stainless steel. However, in view of the electronic coupling between a plug (not shown) inserted in the receptacle 522, and magnets 504, the housing 502 could conceivably be made from a non-conductive material, such as plastic with the plug and magnet 504 providing a conductive path between the current provided to the plug (not shown) and the tool (not shown) to which the magnet 505 is magnetically coupled.

The housing 502 includes a first magnet receiving cavity 504 for receiving a first magnet 505, and a second magnet receiving cavity 506 for receiving a second magnetic member 507. A receptacle receiving cavity 508 extends generally parallel to the long axis of the housing 502, and is drilled, to extend inwardly from the first end surface 514 of the housing 502.

The housing includes an upper surface 510 upon which an indicia, such as the tool provider's name can be embossed and engraved or forged; and a lower surface 512. The lower surface 512 includes the apertures that permit one to gain entrance to the first and second magnet receiving cavities 504, 506. The housing 502 also includes a first end surface 514 into which the receptacle receiving cavity 508 is drilled or formed, and a second end surface 516 disposed in an opposed relation to the first end surface 514. The housing 502 further includes first and second side surfaces 518, 520. A plug receptacle 522 is separately formed from the housing 502 and is provided for insertion therein. The plug receptacle 522 includes a threaded distal portion 524 for threadedly engaging female threads that are formed in the surface of the receptacle receiving cavity 508. The plug receptacle 522 also includes a hex-headed proximal portion 526 that terminates in a plug receiving aperture 528. The hex-headed proximal portion 526 is provided so that one can employ a wrench to turn the plug receptacle member 522 in the receptacle receiving cavity 508, to insert the receptacle member 522 into and remove it from the receptacle receiving cavity 508.

Magnets 505, 507 are insertable in the respective first and second receiving cavities 504, 506. Preferably, the magnets are sized to be press fit into the cavities 504, 506 so that they will remain positioned within the respective cavity without falling out. Otherwise, they can be chemically bonded, or else covered over with a layer that might cover a portion of the bottom surface 512 of the housing 502, to maintain the magnets 505, 507 within the cavities.

Turning now to FIGS. 19A-22B, an alternate embodiment magnetically attachable coupler 600 is shown for use in connection with a therapy tool. The magnetically attachable coupler 600 is, from a functional standpoint, a variant of the embodiment 500 shown in FIGS. 19-22, with the primary difference being that coupler 600 includes three magnet receiving cavities 604, 606, 609, and three magnets 605, 607, 611 for being received in the respective three cavities.

In particular, an electrical source (not shown) is provided that contains a plug (not shown), that is insertable into a plug receptacle 622 of the coupler 600 for providing a source of current to the coupler 600. The current so provided is then transferred to a therapy tool (e.g. tool 90) to which the magnetically attachable coupler 600 is attached. This current is then transferred, through a conductive path of the tool 90, to the patient's skin, and ultimately to subcutaneous tissue, such as scar tissue adhesions, upon which the current can act in a therapeutic manner.

The coupler 600 includes a housing 602 that is preferably made from a current conducting, magnetizable material such as stainless steel. However, in view of the electronic coupling between a plug (not shown) inserted in the receptacle 622, and magnets 604, the housing 602 could conceivably be made from a non-conductive material, such as plastic with the plug and magnet 604 providing a conductive path between the current provided to the plug (not shown) and the tool (not shown) to which the magnet 605 is magnetically coupled.

The housing 602 includes a first magnet receiving cavity 604 for receiving a first magnet 605, and a second magnet receiving cavity 606 for receiving a second magnetic member 607, and a third magnet receiving cavity 609 for receiving a third magnet 611 A receptacle receiving cavity 608 extends generally parallel to the long axis of the housing 602, and is drilled, to extend inwardly from the first end surface 514 of the housing 602.

The housing includes an upper surface 610 upon which an indicia, such as the tool provider's name can be embossed and engraved or forged; and a lower surface 612. The lower surface 612 includes the apertures that permit one to gain entrance to the first and second magnet receiving cavities 604, 606. The housing 602 also includes a first end surface 614 into which the receptacle receiving cavity 608 is drilled or formed, and a second end surface 616 disposed in an opposed relation to the first end surface 614. The housing 602 further includes first and second side surfaces 618, 620.

A plug receptacle 622 is separately formed from the housing 602 and is provided for insertion therein. The plug receptacle 622 includes a threaded distal portion 624 for threadedly engaging female threads that are formed in the surface of the receptacle receiving cavity 608. The plug receptacle 622 also includes a hex-headed proximal portion 626 that terminates in a plug receiving aperture 628. The hex-headed proximal portion 626 is provided so that one can employ a wrench to turn the plug receptacle member 622 in the receptacle receiving cavity 608, to insert the receptacle member 622 into and remove it from the receptacle receiving cavity 608.

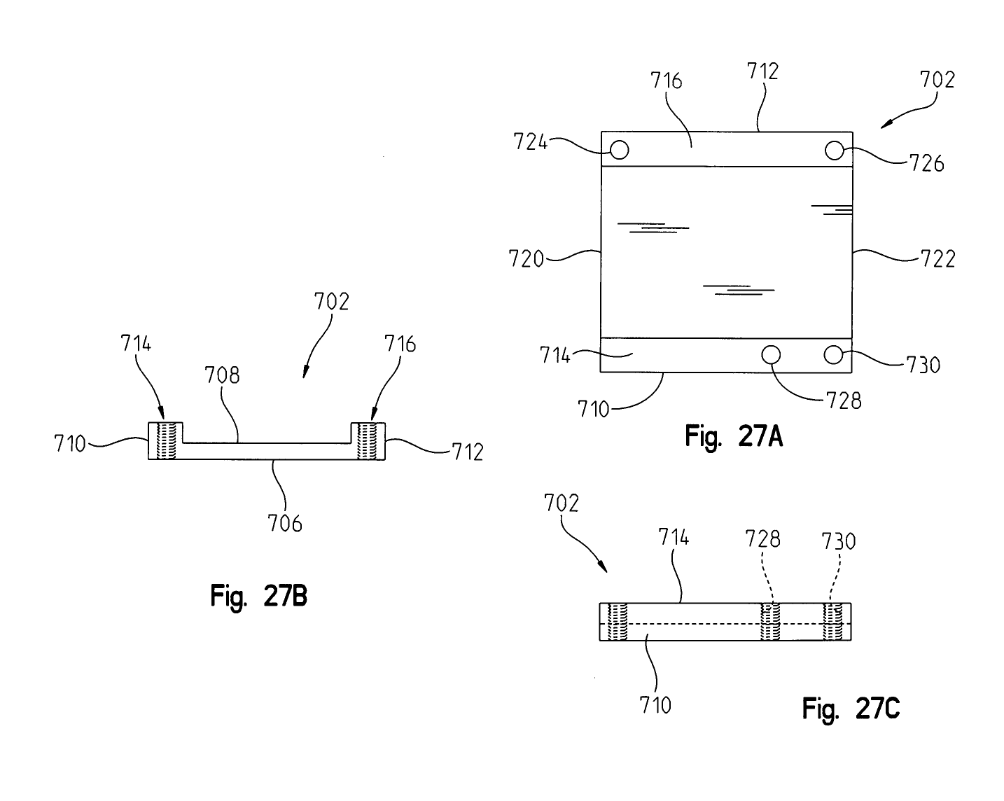

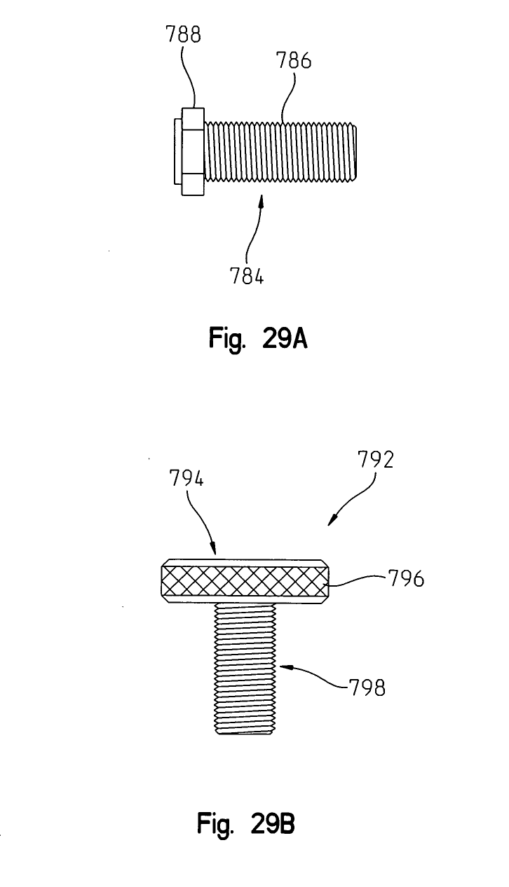

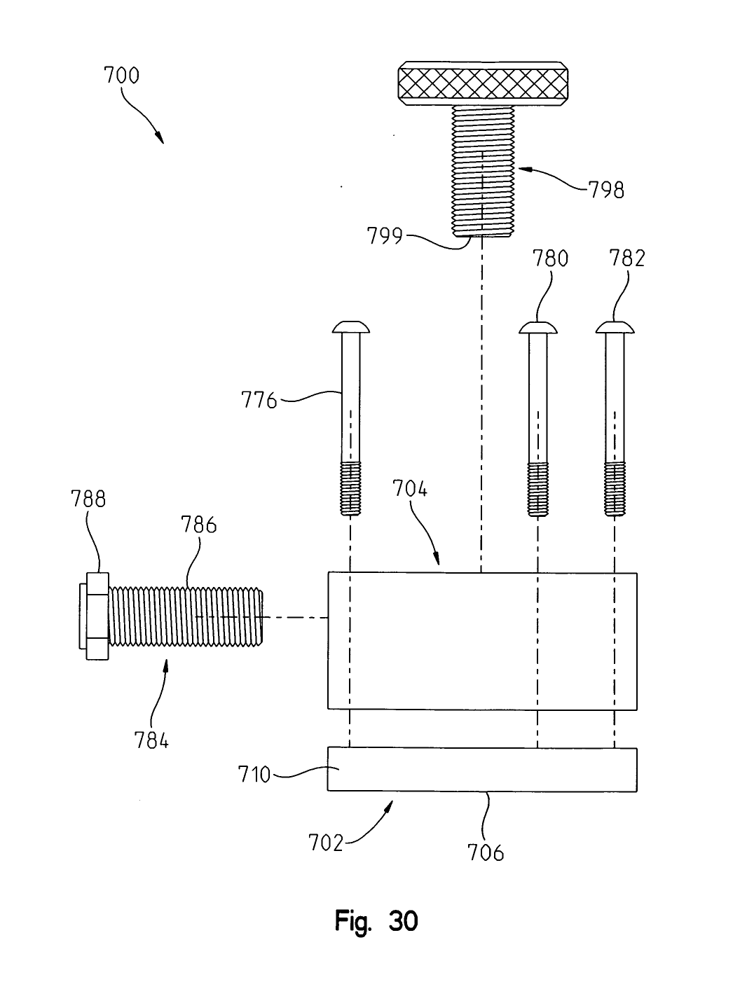

Magnets 605, 607 are insertable in the respective first and second receiving cavities 604, 606, 609. Preferably, the magnets 605, 607, 609 are sized to be press fit into the cavities 604, 606, 608 so that they will remain positioned within the respective cavity without falling out. Otherwise, they can be chemically bonded, or else covered over with a layer that might cover a portion of the bottom surface 612 of the housing 602, to maintain the magnets 605, 607, 611 within the cavities. A connector 700 that employs a clamp for clamping the connector 700 on to a tool (not shown) is shown in FIGS. 27A-30. The connector 700 is somewhat similar to the connector shown in FIGS. 10-13, and comprises a two-piece connector that includes a lower member 702 and an upper member 704 that are joined together to form a single connector 700. The lower member 702 is shown best in FIGS. 27A-27C and FIG. 30, as including a bottom surface 706 and an upper (interior) surface 708, a first side surface 710 and a second side surface 712. The lower member 702 also includes first and second upstanding walls 714, 716, a first end surface 720 and a second end surface 722.

Four screw receiving apertures 724, 726, 728, 730 are preferably designed to be threaded, and formed in the upper surface of the walls 714, 716 for receiving screws 776 (not shown), 780, 782 that are shown in FIG. 30, and are employed for coupling together the upper and lower members 704, 702.

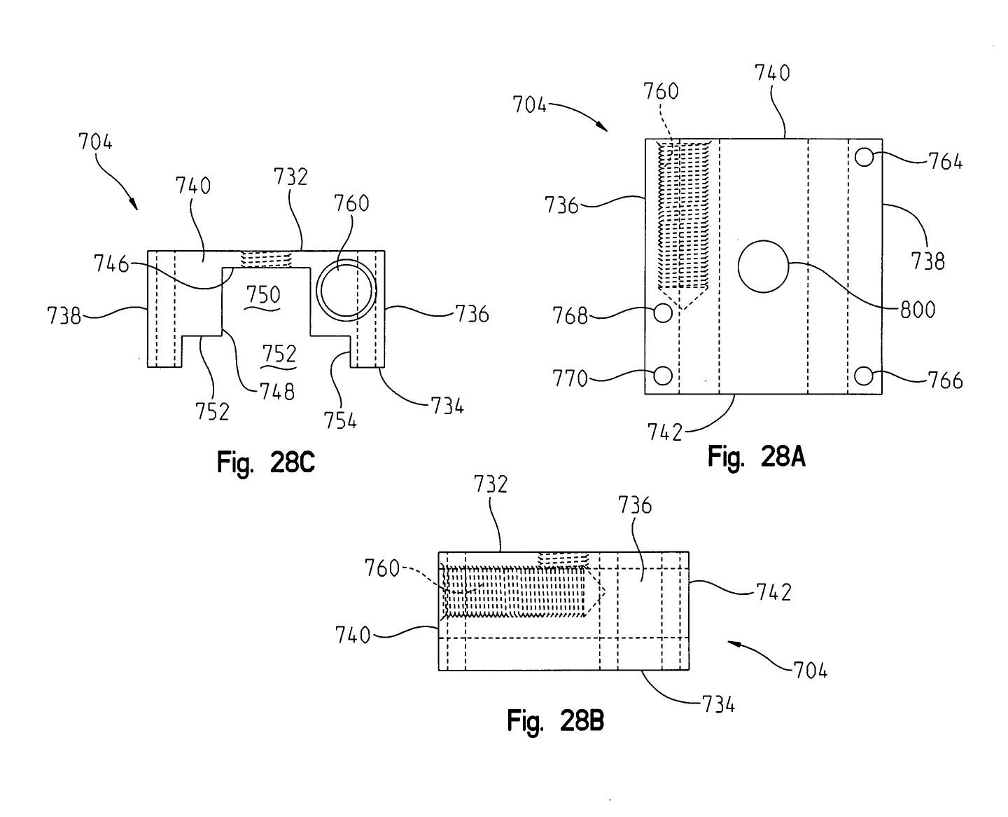

Preferably, each of the upper and lower members 704, 702 are formed from an electrically conductive material such as stainless steel. The upper member 704 is best shown in FIGS. 28A-28C and FIG. 30 as including an upper surface 732 and a lower surface 734. The lower surface 734 also comprises an interior surface. The upper member 704 further includes a first side surface 736, a second side surface 738, a first end surface 740 and a second end surface 742.

The lower surface 734 is not planar, but rather, is formed into several different segments. The lower surface 734 includes a first, generally planar relatively horizontally disposed portion 746 that terminates at its side in a pair of relatively vertically disposed wall portions 748. The first wall portions 748, along with the horizontal portions 746 define a first, relatively narrower passageway portion 750 of the assembled connector 700. The wall portions 748 at their lowest extreme, terminate at relatively horizontally extending shoulder portions 742.

The shoulder portions 742 terminate at their outward ends at a pair of second wall portions 754. The shoulder portions 752, second wall portion 754, and the upper surface 708 of the lower member 702 together define the relatively wide passageway portion 752 that is sized and positioned for receiving a tool member, such as tool member 90. It will be appreciated that when a tool is inserted into the passageway, the shoulder portions 752 will be positioned above the upper surface of the tool, and the second wall portions 754 will be positioned adjacent to the side surfaces of the tool 90.

In this configuration, the shoulder portions, in combination with the second wall portion 754 and the upper surface 708 help to restrict the movement of the tool in the passageway. Because of their positioning, the shoulders 752 help to prevent the tool from being lifted up, as they restrict the vertical movement of the tool 90.

A receptacle receiving cavity 760 is drilled from, and extends inwardly from the first end surface 740 of the upper portion 704. The receptacle receiving cavity 760 preferably includes female threads for receiving the threaded distal end 768 of the receptacle member 784.

First, second, third and fourth screw receiving apertures 764, 766, 768, 770 extend through the upper portion 704, so that attachment screws 776 (not shown) 780, 782 can be inserted respectively in the first, second, third and fourth screw receiving apertures 764, 766, 768, 770, so that the upper member 704 can be securely coupled to the lower member 702. The screw receiving apertures 764-770 are positioned to line up with the screw receiving apertures 724, 726, 728, 730 of the lower member, so that the lower ends of the threaded screws 776 (not shown), 780, 782 can be threadedly received within the threaded apertures 724-730 of the lower member 702.

The plug receptacle 784 includes a threaded distal end portion 786 for being threadedly received within the receptacle receiving cavity 760. The plug receptacle 784 also includes a hex-shaped proximal portion 788, for enabling the user to tighten the plug receptacle 786 in the receptacle receiving cavity 760 by engaging a wrench with the hex-shaped end 788.

The screw clamp 792 is provided for being movable relative to the upper and lower members 702, 704 of the connector so that the screw clamp 792 can move between a tool engaging position, wherein the distal end 799 of the post 798 can engage the upper surface of the tool; and a tool disengaged position. In the tool disengaged position, the distal end 799 does not engage the tool, or otherwise, engages it lightly to permit the tool to be removed from its coupling to the connector 700.

The screw clamp 792 includes a head portion 794 that is generally disc-shaped and includes a knurled circumferential surface 796. The circumferential surface 796 is knurled to improve the user's ability to grip the screw clamp to rotate it between a tool engaged and a tool disengaged position. A threaded post 798 extends axially along a line generally perpendicular to the major plane of a head portion 794. The threads of the threaded post 798 are sized and positioned to engage the threads of the threaded screw clamp receiving aperture 800 that is formed in the upper member 704 so that rotary movement of the clamp 792 moves the axial position of the clamp 792.

Having described the invention with reference to certain detailed embodiments, it will be appreciated that the scope and spirit of the invention extend far beyond the embodiments described herein, and are limited only by the prior art.

What is claimed is:

1. A therapy tool system comprising
a rigid therapy tool for passing across a patient's skin for loosening subcutaneous tissue, the tool including an electrically conductive portion engagable with a patient's skin,
a current source for providing an electrical current,
a connector member selectively attachable to and removable from the rigid tool, the connector member being coupled to the current source for conducting current between the current source and the rigid tool,
wherein the rigid tool includes a conductive path for conducting current from the connector to the electrically conductive portion of the tool, to enable electrical current delivered to the connector member from the current source to be conducted to the electrically conductive portion of the rigid tool and transferred from the rigid tool through the skin to subcutaneous patient tissue.

2. The therapy tool of claim 1 wherein the connector member includes a sleeve portion for receiving the rigid tool, and a clamp for fixedly positioning the connector with respect to the tool, while also permitting ready detachment of the connector from the rigid tool, and wherein the subcutaneous tissue comprises fibrous scar tissue underlying patient soft tissue.

3. The therapy tool of claim 1 wherein the current source includes a plug member and the connector includes a plug receptacle for engagement with the plug member.

4. The therapy tool of claim 3 wherein the plug member is generally cylindrical and the plug receptacle includes a generally cylindrical female aperture for matingly snugly receiving the cylindrical member portion.

5. The tool system of claim 3 wherein the sleeve portion includes a relatively wider portion for receiving the rigid tool, and a relatively narrower portion, the relatively narrower portion being defined by shoulders for movement of a total received in the relatively wide portion.

6. The tool system of claim 5 wherein the clamp includes a threaded member axially movable with respect to the connector between a tool engaging and a tool releasing position.

7. The tool system of claim 6 wherein the rigid tool includes a metal body portion that includes a metal edge, the metal body portion serving as the conductive path for conducting current from the connector to the electrically conductive edge for transferring current from the rigid tool to subcutaneous patient tissue.

8. The therapy tool of claim 1 wherein the connector member includes a body defining a sleeve for receiving the rigid tool, and a clamp member movable between a tool engaging and tool releasing position.

9. The therapy tool of claim 8 wherein the body includes shoulders for at least partially defining the sleeve, the shoulders being positioned for limiting the movement of the tool.

10. The therapy tool of claim 8 wherein the body includes a threaded clamp receiving aperture, and the clamp includes a threaded member for threadedly engaging the threaded aperture and wherein the clamp is movable relative to the body between a tool engaging position and tool release position.

11. The therapy tool of claim 1 wherein the connector member includes a body defining a sleeve portion for receiving the rigid tool and a clamp, the clamp comprising a screw clamp movable between a tool release position wherein the rigid tool can be inserted into and removed from the sleeve, and a tool engaging position wherein the clamp fixedly positions and couples the connector to the rigid tool.

12. The therapy tool of claim 11 wherein the sleeve portion holds the tool in a plane and the clamp comprises a screw clamp axially movable along a line generally perpendicular to the plane.

13. The therapy tool of claim 1 wherein the rigid tool includes a body, the body including side walls, a bottom wall and shoulder members that define a sleeve having a relatively wide tool receiving portion and a relatively narrower portion, wherein the shoulders are provided for restricting movement of the rigid tool to maintain the rigid tool in the relatively wider portion.

14. The therapy tool of claim 1, wherein the rigid tool includes a first rigid tool and a second rigid tool, and the connector is movable between the first rigid tool and the second rigid tool.

15. The rigid tool of claim 14 wherein connector includes a body that defines a sleeve portion and a clamp movable between a tool engaging portion and a tool releasing position, wherein the sleeve is sized and configured to receive each of the first and second rigid tools, and the clamp is sized and positioned to engage each of the first and second rigid tools.

16. The therapy tool of claim 1 wherein the rigid tool includes a metal body portion that includes a metal edge, the metal body portion serving as the conductive path for conducting current from the connector to the electrically conductive edge for transferring current from the rigid tool to subcutaneous patient tissue.

17. The therapy tool of claim 1 wherein the current source includes a plug member and the connector includes a cavity-like plug receptacle for interiorly receiving the plug member.

18. The therapy tool of claim 1 wherein the connector member comprises a magnetic member for being magnetically and electrically coupled to the rigid tool.

19. The therapy tool of claim 18 wherein the connector member includes a plug receptacle for receiving a plug, and the current source includes a plug member capable of being coupled to the plug receptacle for delivering current from the current source to the connector.

20. The therapy tool of claim 19 wherein the connector member comprises a magnetic metal body having a generally rectangularly cuboid shape and a long axis, and the plug receptacle comprises a generally cylindrical interior passageway extending generally parallel to the long axis of the metal body.
